(19) United States
(12) Patent Application Publication
(10) Pub. No.: US 2012/0094974 A1
(43) Pub. Date: Apr. 19, 2012

(54) SMOOTHENED RECEPTOR MODULATORS

(76) Inventors: Wei Chen, Durham, NC (US); Lawrence Barak, Durham, NC (US); H. Kim Lyerly, Durham, NC (US); Jiangbo Wang, Durham, NC (US)

(21) Appl. No.: 12/737,044

(22) PCT Filed: Jun. 16, 2009
(86) PCT No.: PCT/US2009/003604

§ 371 Date: Jun. 21, 2011

Related U.S. Application Data
(60) Provisional application No. 61/129,302, filed on Jun. 17, 2008.

(51) Int. Cl. A61K 31/454; A61K 31/5377; A61K 31/55; A61K 31/553; A61P 35/00; A61K 31/551; A61K 31/4545; A61K 31/426; A61K 31/4439; A61K 31/538; A61K 31/506; A61K 31/4725

(52) U.S. Cl. 514/210.21; 514/252.18; 514/252.14; 514/235.5; 514/318; 514/217.04; 514/211.15; 514/307; 514/218; 514/365; 514/342; 514/230.5

(57) ABSTRACT

The present invention relates, in general, to the Smoothened receptor and, in particular, to a method of modulating Smoothened receptor activity and to compounds and compositions suitable for use in such a method.

This application claims priority from U.S. Provisional Application No. 61/129,302, filed Jun. 17, 2008, the entire content of which is incorporated herein by reference.

This invention was made with government support under RO1 CA 113656-O1A1 awarded by the National Institutes of Health. The government has certain rights in the invention.

### TECHNICAL FIELD

The present invention relates, in general, to the Smoothened receptor and, in particular, to a method of modulating Smoothened receptor activity and to compounds and compositions suitable for use in such a method.

### BACKGROUND

Hedgehog (Hh) signaling is mediated by regulation of a protein called Smoothened (Smo) that spans the cell membrane seven times (7MS), activation of which sets in motion transcriptional events that control growth and patterning in vertebrate development (Cohen Jr., Am. J. Med. Genet. 123A:5 (2003)), Ingham et al, Genes Dev. 15:3059 (2001)). Dysregulated Smo activity leads to several forms of cancer (Xie et al, Nature 391:90 (1998), Wechsler-Reya et al, Annu. Rev. Neurosci. 24:385 (2001), Berman et al, Nature 425:846 (2003), Watkins et al, Nature 422:313 (2003), Thayer et al, Nature 425:851 (2003)).

Hh binds to a receptor, Patched (Ptc), that spans the cell membrane 12 times and relieves inhibitory control of Smo by Ptc. However, almost nothing is known of the mechanisms operating just downstream of Smo to mediate and modulate its actions.

β-Arrestins are cytosolic proteins that bind to most activated 7MS receptors after the receptors have been phosphorylated by G protein-coupled receptor kinases (GRKs), which promotes internalization of the receptors and some forms of signaling (Luttrell et al, J. Cell Sci. 115:455 (2002), Pitcher et al, Annu. Rev. Biochem. 67:653 (1998)). It has been demonstrated that β-arrestin (βarr2) and GRK2 mediate clathrin-dependent internalization of Smo (Chen et al, Science 306:2257 (2004)). However, it is possible that they may also modulate or mediate aspects of Smo signaling, as is the case for other 7MS receptors (Luttrell et al, J. Cell Sci. 115:455 (2002), Pitcher et al, Annu. Rev. Biochem. 67:653 (1998), McDonald et al, Science 290:1574 (2000), Luttrell, J. Mol. Endocrinol. 30:117 (2003)). Indeed, β-arrestin 2 knockdown in zebrafish embryos by morpholino antisense leads to a lethal developmental phenotype (Wilbanks et al, Science 306:2264 (2004)) that is remarkably similar to that seen after genetic knockouts of either Smo or Gli2 (van Eeden et al, Development 123:153 (1996), Barresi et al, Development 127:2189 (2000), Chen et al, Development 128:2385 (2001)).

Although Smo is reported to activate Gαᵢ directly or indirectly in frog melanophores (DeCamp et al, J. Biol. Chem. 275:26322 (2000)), no genetic evidence to support coupling of Smo to G proteins has been reported. Several cytosolic components downstream of Smo, such as Costal2 (Cos2), Fused (Fu), Suppressor of Fused, and Cubitus interruptus (Ci), have been identified in Drosophila, and the protein complex containing Cos2, Fu, and Ci has been reported recently to associate with Smo via Cos2 (Ruel et al, Nature Cell Biol. 5:907 (2003), Ogden et al, Curr. Biol. 13:1998 (2003), Jia et al, Genes Dev. 17:2709 (2003), Lum et al, Mol. Cell. 12:1271 (2003)). However, βarr2 and GRK2 interact with mammalian Smo in an activation-dependent manner and, thus, provide a platform for development of screening assays to identify ligands that regulate the activity of this oncogenic receptor and that can be expected to be useful as therapeutic agents.

The present invention relates to ligands that modulate Smo activity and to methods of using such ligands in cancer treatment and other therapeutic settings.

### SUMMARY OF THE INVENTION

The present invention relates generally to the Smo receptor. More specifically, the invention relates to a method of modulating Smo receptor activity and to compounds and compositions suitable for use in such a method.

Objects and advantages of the present invention will be clear from the description that follows.

### BRIEF DESCRIPTION OF DRAWINGS

FIGS. 1A-1W. Structures of compounds tested for affinity to Smo. Supplier ID is the TRIPOS catalog number and the compound identification number given in the TRIPOS database.

FIGS. 2A-2I. Smo receptor binding data for compounds depicted in FIG. 1. In house ID number. The scaffolds for each of the compound families is also shown. The number in the left column is the in-house number assigned to the indicated compound. The number given in the column headed “Binding” is the relative binding ability of the indicated compound compared to cyclopamine, where 1=poor/none and 0=good. The number given in the column headed “Gli” is the relative inhibition of Gli activity where 1=poor/none and 0=good. The “Binding” values were determined were using the assay described in Example 2. The “Gli” values were determined using a separate Gli reporter assay (Corbit et al, Nature 437 (7061):1018-1021 (2005)). Preferred compounds have “Binding” and “Gli” values less than 0.5, more preferred compounds have “Binding” and “Gli” values less than 0.25.

### DETAILED DESCRIPTION OF THE INVENTION

The present invention relates to compounds that modulate Smo activity (e.g., that are Smo activity antagonists) and to compositions comprising same. The invention further relates to the use of such compounds and compositions in various therapeutic settings, including cancer treatment, wound repair and tissue regeneration.

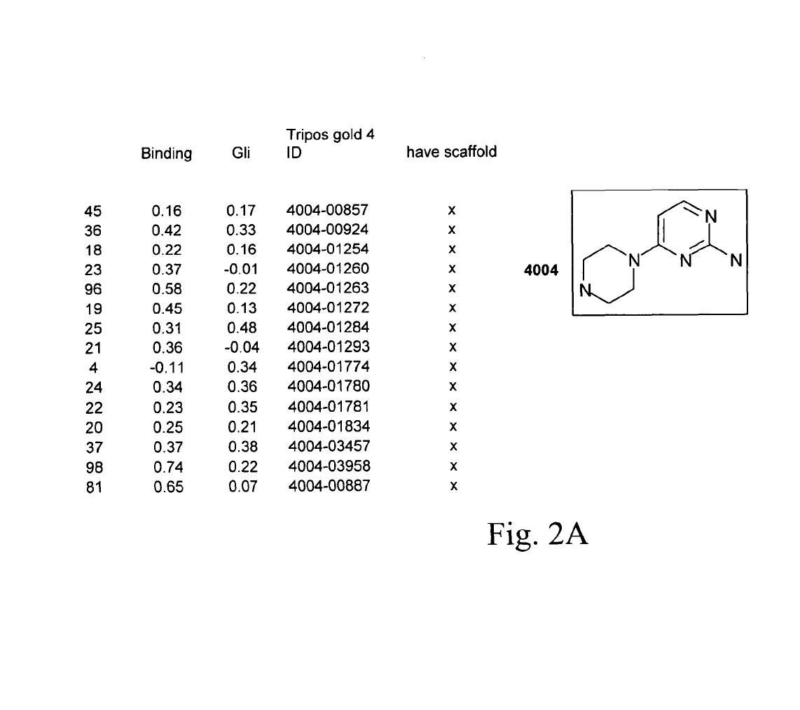

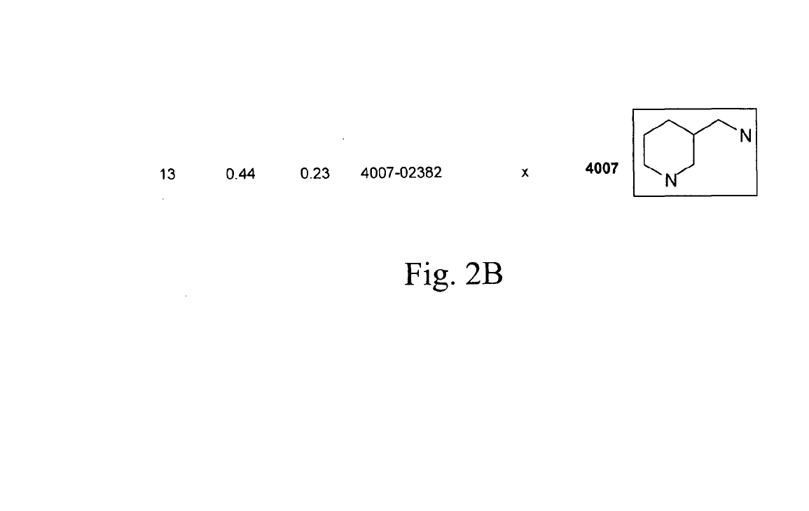

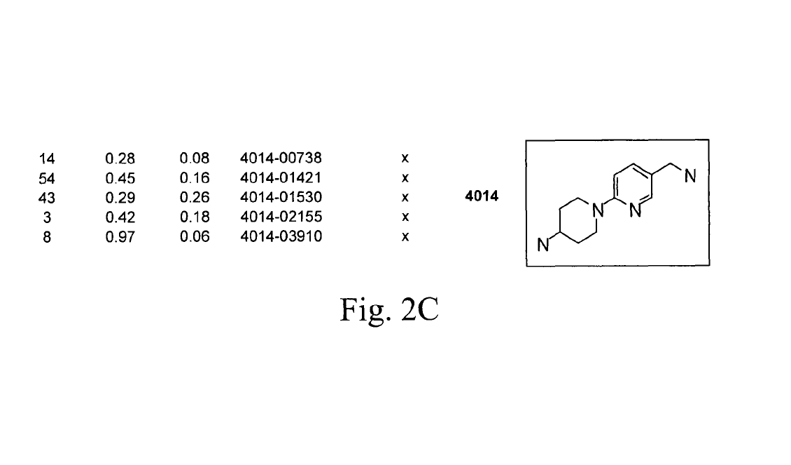

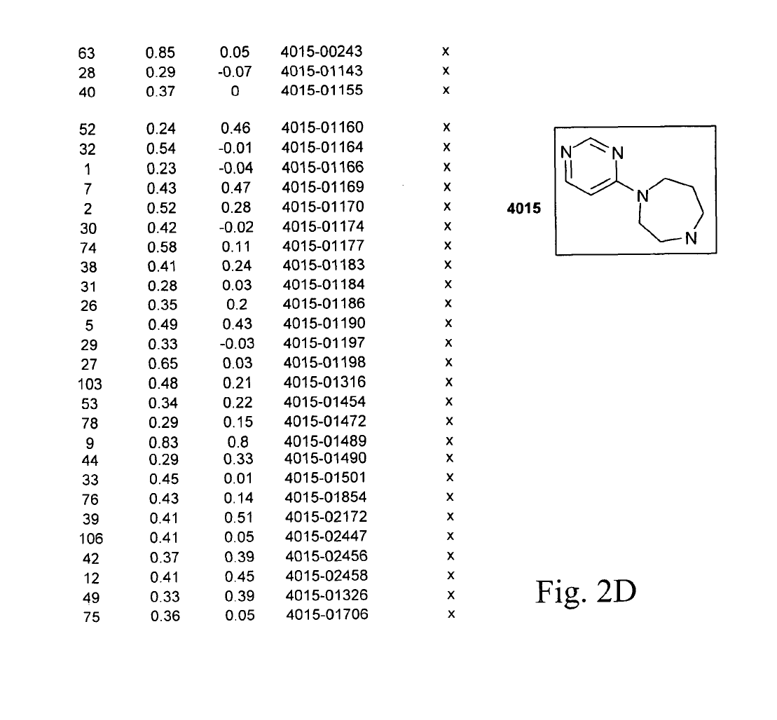

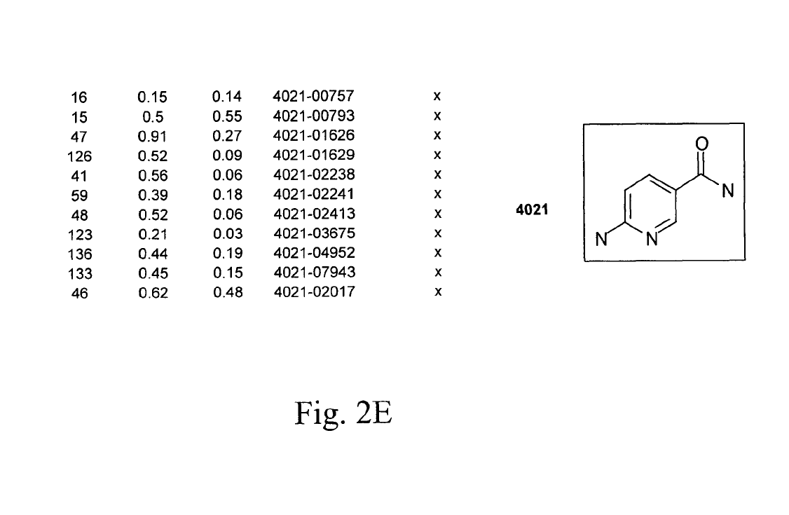

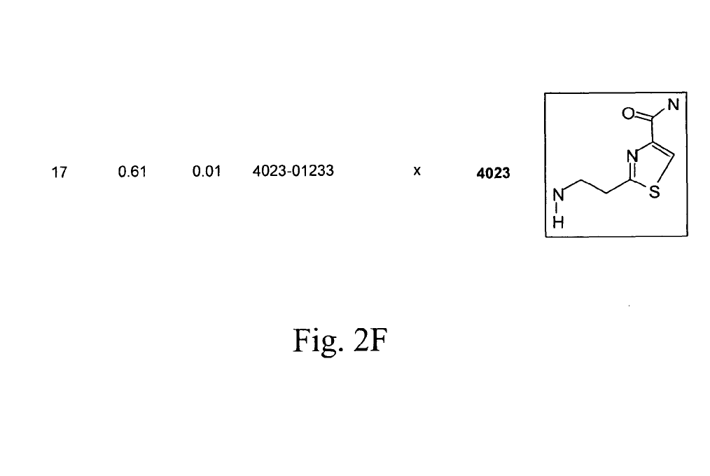

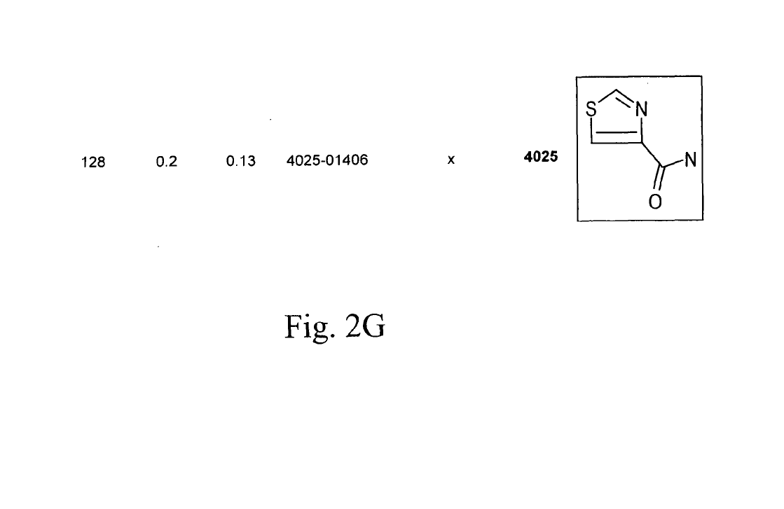

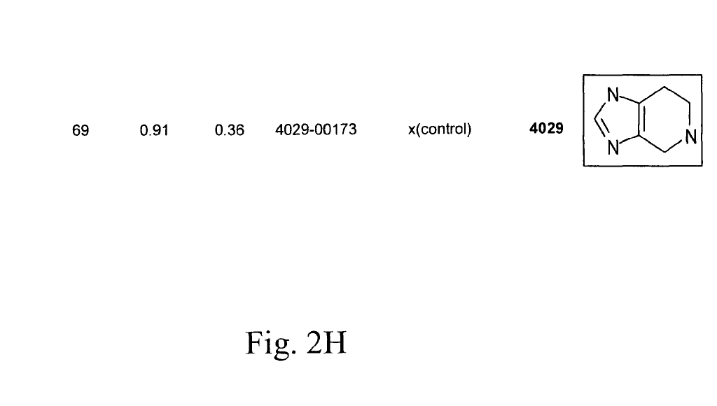

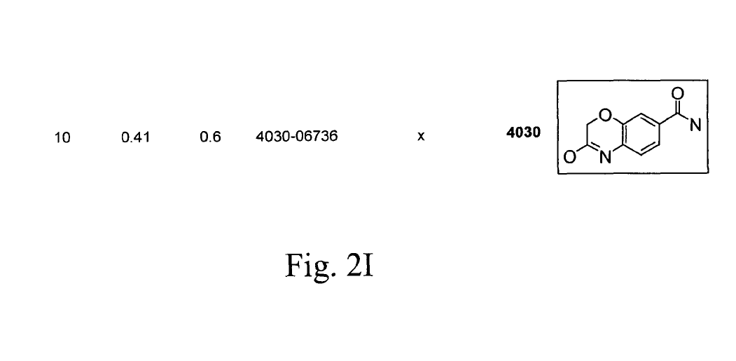

In one embodiment, the invention relates to compounds of formula I (see scaffold 4004 in FIG. 2):

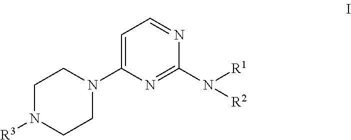

wherein R¹ is an linear or branched alkyl (preferably, a C₁-C₄ alkyl, more preferably, methyl) and R² is —(CH₂)ₙ-aryl (preferably

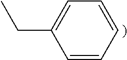

or —(CH₂)ₙ—heteroaryl (preferably,

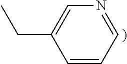

and
n=1 to 4 (preferably 1 or 2, more preferably 1)
or R¹ and R² together with the nitrogen to which they are attached are

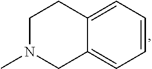

and
R³ is

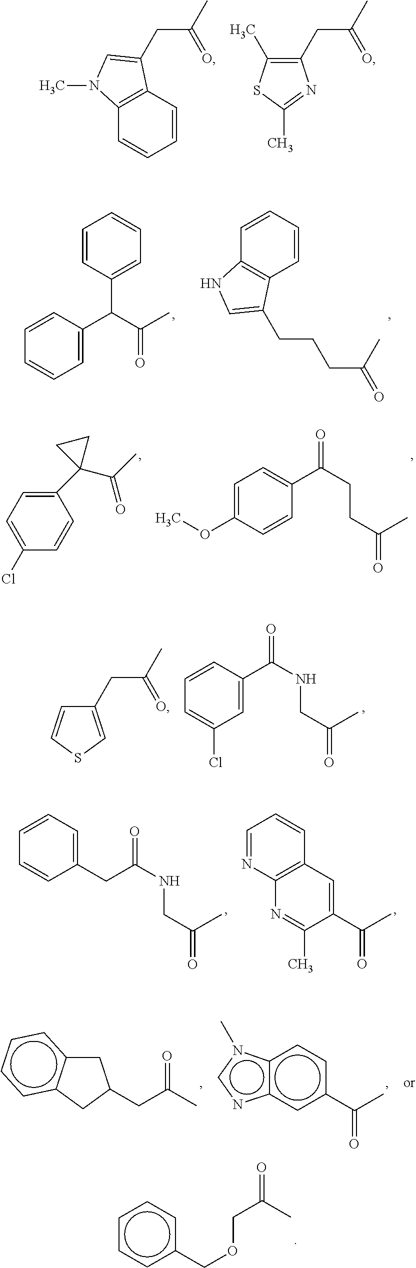

In a further embodiment, the invention relates to compounds of formula II (see scaffold 4007 in FIG. 2):

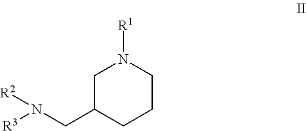

wherein R¹ is

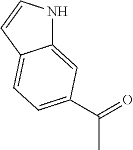

and R² and R³, together with the nitrogen

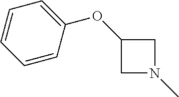

to which they are attached are

In yet another embodiment, the invention relates to compounds of formula III (see scaffold 4014 in FIG. 2):

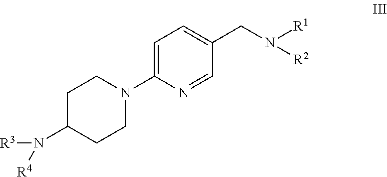

wherein R¹ and R², together with the nitrogen to which they are attached, are

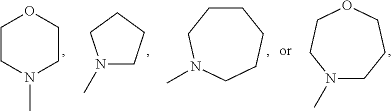

or
R¹ and R² are alkyl (preferably, C₁-C₄ alkyl, more preferably R¹ is methyl and R² is ethyl), and
R³ is H and R⁴ is

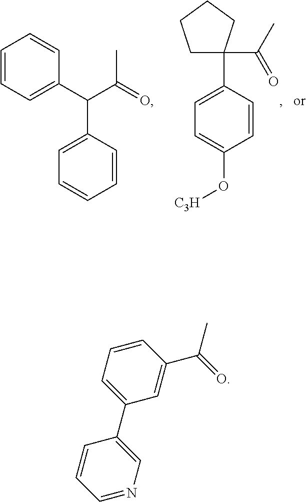

In a further embodiment, the invention relates to compounds of formula IV (see scaffold 4015 in FIG. 2):

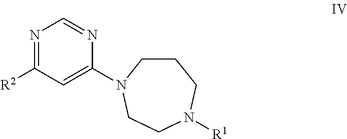

wherein R¹ is

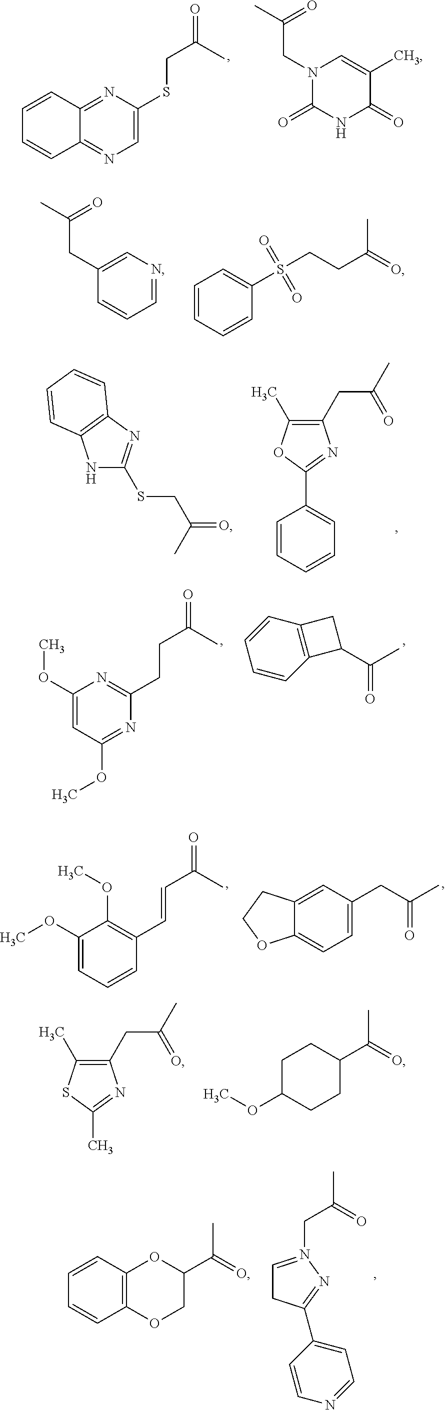

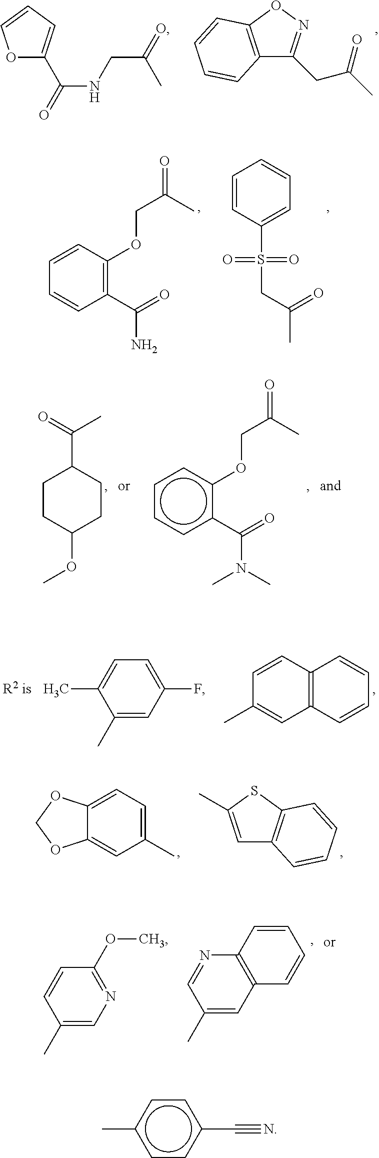

In yet another embodiment, the present invention relates to compounds of the formula V (see scaffold 4021 in FIG. 2):

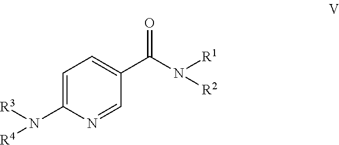

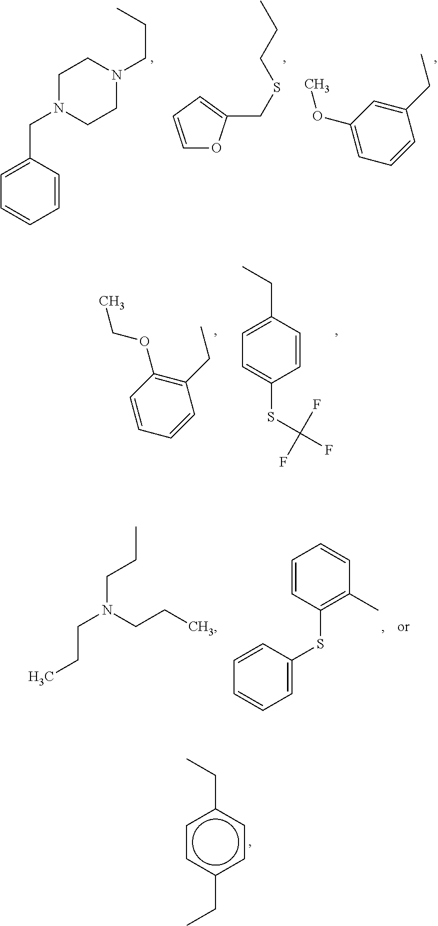

wherein R¹ is H and R² is
   or
R¹ and R² and the nitrogen to which they are attached is

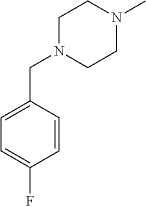

and
R³ and R⁴ and the nitrogen to which they are attached is

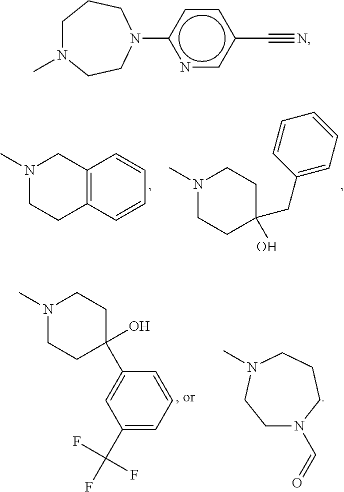

In a further embodiment, the invention relates to a compound of formula VI (see scaffold 4023 in FIG. 2):

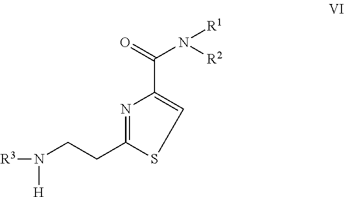

wherein R¹ is H and R² is

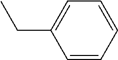

and
R³ is

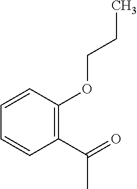

In a further embodiment, the present invention relates to compounds of formula VII (see scaffold 4025 in FIG. 2):

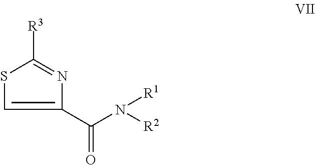

wherein R¹ is CH₃, and
R² is

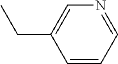

### and R³ is

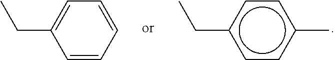

In yet another embodiment, the present invention relates to compounds of formula VIII (see scaffold 4030 in FIG. 2):

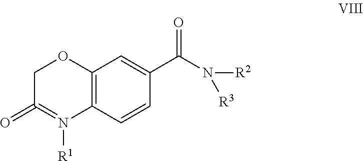

wherein R¹ is

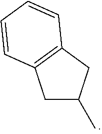

### R² is H and R³ is

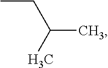

The invention includes pharmaceutically acceptable salts of the above compounds, as may be appropriate.

Preferred compounds have the greatest effect on targeting Gli activity and/or cyclopamine binding, or in the primary β-arrestin assay (Chen et al, Science 306:2257 (2004)) for blocking translocation.

The compounds identified above can be used in method of treating cancers in human and non-human animals. Cancers amenable to treatment include, but are not limited to, adenocarcinomas of the pancreas, prostate, breast, stomach, esophagus and biliary tract; medulloblastomas and gliomas; small-cell lung cancers; basal cell carcinomas; rhabdomyosarcomas; urothelial carcinomas; squamous cell carcinomas of the oral cavity; and hepatocellular carcinomas. Optimum dosing regimens and suitable routes of administration (e.g., oral, topical or IV) can be determined by one skilled in the art and can vary with the compound, the patient and the effect sought.

Compounds of the invention can control a pathway important for organ differentiation, including the gastrointestinal tract, skin and brain. Therefore, titration of smoothened agonists or antagonists can be used responsively to correct errors in growth and differentiation that may arise during the prenatal period or to augment different stages of cellular repair that may occur during periods of tissue regeneration.

Compounds described above can be formulated into pharmaceutical compositions suitable for use in the present methods. Such compositions include the active agent, together with a pharmaceutically acceptable carrier, excipient or diluent. The composition can be present in dosage unit form, for example, tablets, capsules or suppositories. The composition can also be in the form of a sterile solution suitable for injection or nebulization. Compositions can also be in a form suitable for opthalmic use. The invention also includes compositions formulated for topical administration, such compositions taking the form, for example, of a lotion, cream, gel or ointment. The concentration of active agent to be included in the composition can be selected based on the nature of the agent, the dosage regimen and the result sought.

The dosage of the composition of the invention to be administered can be determined without undue experimentation and will be dependent upon various factors, including the nature of the active agent, the route of administration, the patient, and the result sought to be achieved. A suitable dosage of a compound of the invention to be administered (e.g., orally, IV or topically) can be expected to be in the range of about 0.01 to 500 mg/kg/day, preferably, 1.0 to 10 mg/kg/day. Suitable doses of compounds can vary, for example, with the compound, the patient and with the result sought.

Certain aspects of the invention can be described in greater detail in the non-limiting Examples that follows. (Incorporated herein by reference, in its entirety, is the application of Chen et al entitled “Radiolabeled Cyclopamine Assay For The Smoothened Receptor”, filed Jun. 17, 2008, Attorney Docket No. DUKE001 (U.S. Provisional Application No. 61/073,250), and PCT application of Chen et al entitled “Radiolabeled Cyclopamine Assay For The Smoothened Receptor”, filed Jun. 16, 2009, Attorney Docket No. DUKE001-PCT.)

### Example 1

### Tritiated Cyclopamine Binding

Cells (U2OS) permanently expressing approximately 10 picomoles per milligram of human Smo receptor were plated at 125,000 cells per well in a 12 well tissue culture plate in Minimal Essential Medium with 10% fetal bovine serum in a 5% CO₂ incubator. The following day, the media was replaced with 100 μl cold phosphate buffered saline (PBS) at pH 7.2 following three washes in PBS. Tritiated cyclopamine was added in 50 μl of cold PBS to each well at varying concentrations and the plate was incubated over ice for ninety minutes on a cell rocker. For controls, duplicate wells containing tritiated cyclopamine and an excess of cold cyclopamine, at 20 micromolar, were also prepared and incubated under the same conditions. Following the incubation, the cells were washed three times in cold PBS and 100 μl of 0.1M NaOH was added to each well for 30 to 60 minutes to extract the remaining bound tritiated cyclopamine. The extracted material was added to a vial containing 2 ml of scintillation fluid. Duplicate or triplicate wells were assayed for each curve. Any cell type expressing the Smo receptor (human or nonhuman) can be used as described above, as can any membrane in which the receptor has been expressed artificially or naturally.

### Example 2

### Tritiated Cyclopamine Assay for Competition Binding of the Smo Receptor

The assay described in Example 1 was used to evaluate the affinity to Smo of the compounds SANT1 and SANT2 (antagonists), cold cyclopamine, the cyclopamine derivatives KAAD-cyclopamine and jervine, and the Smo antagonists SAG1 (Alexis Biochemical, ALX 270-426).

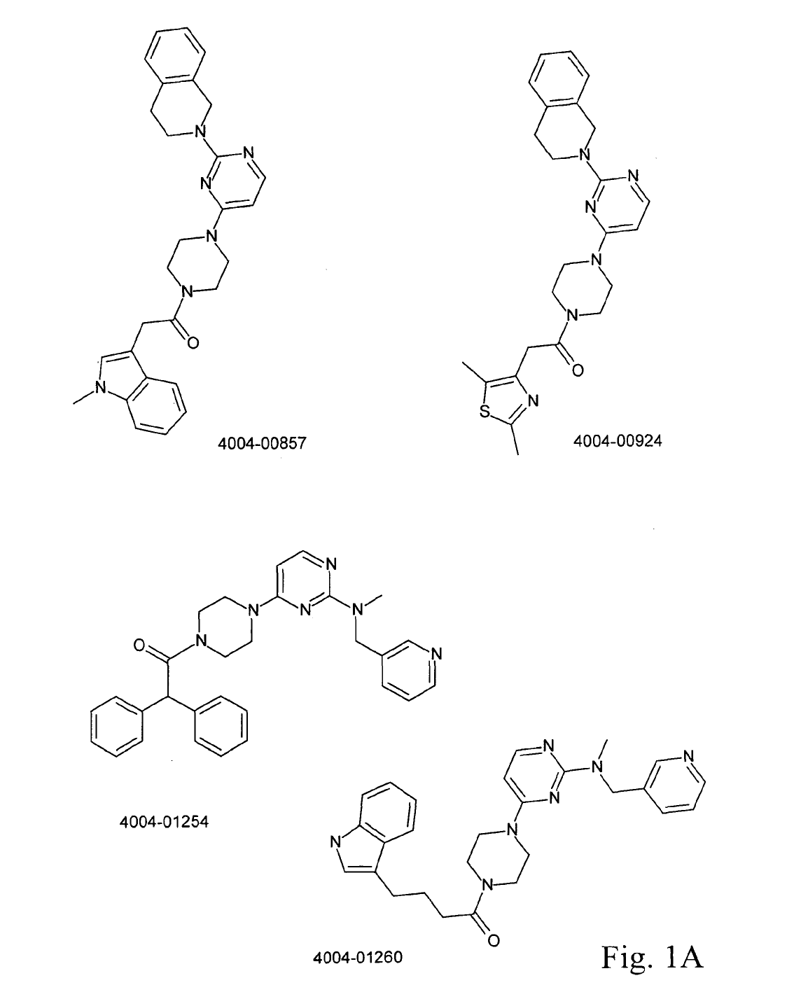

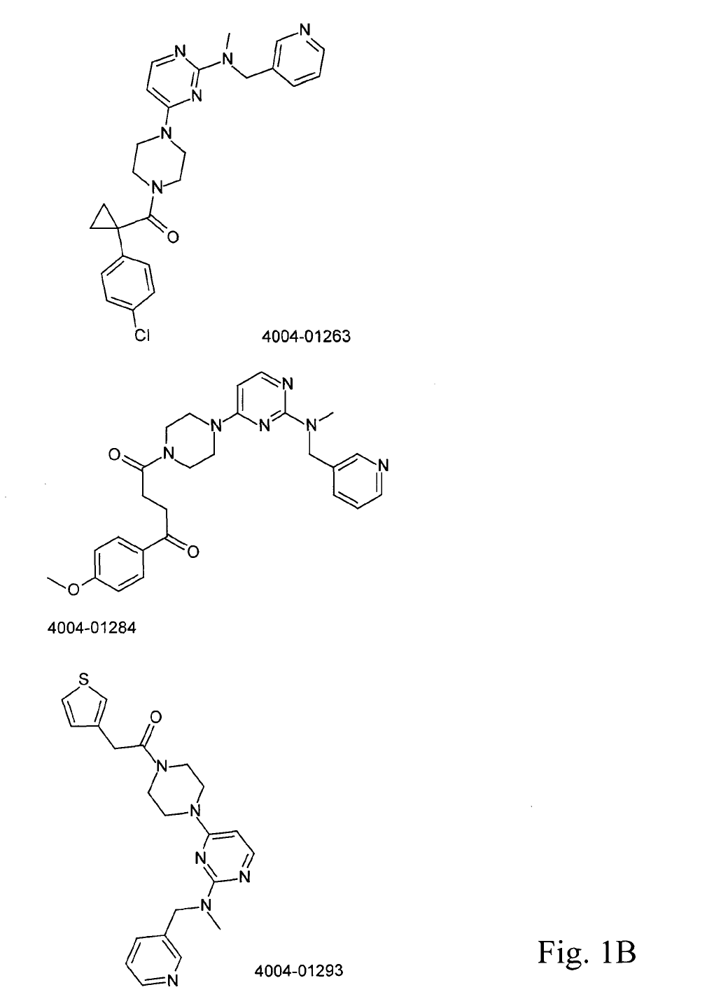

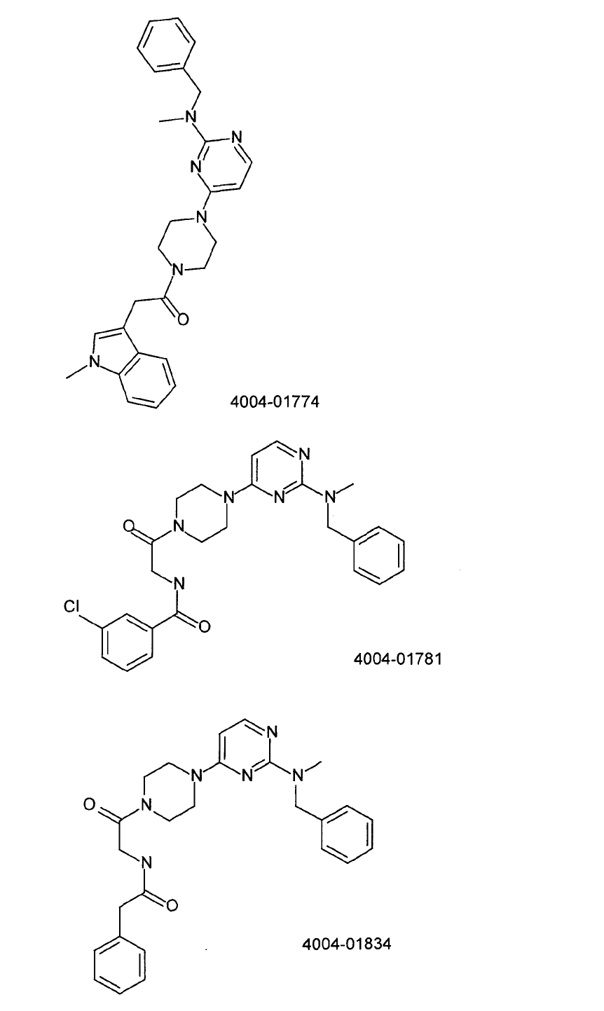

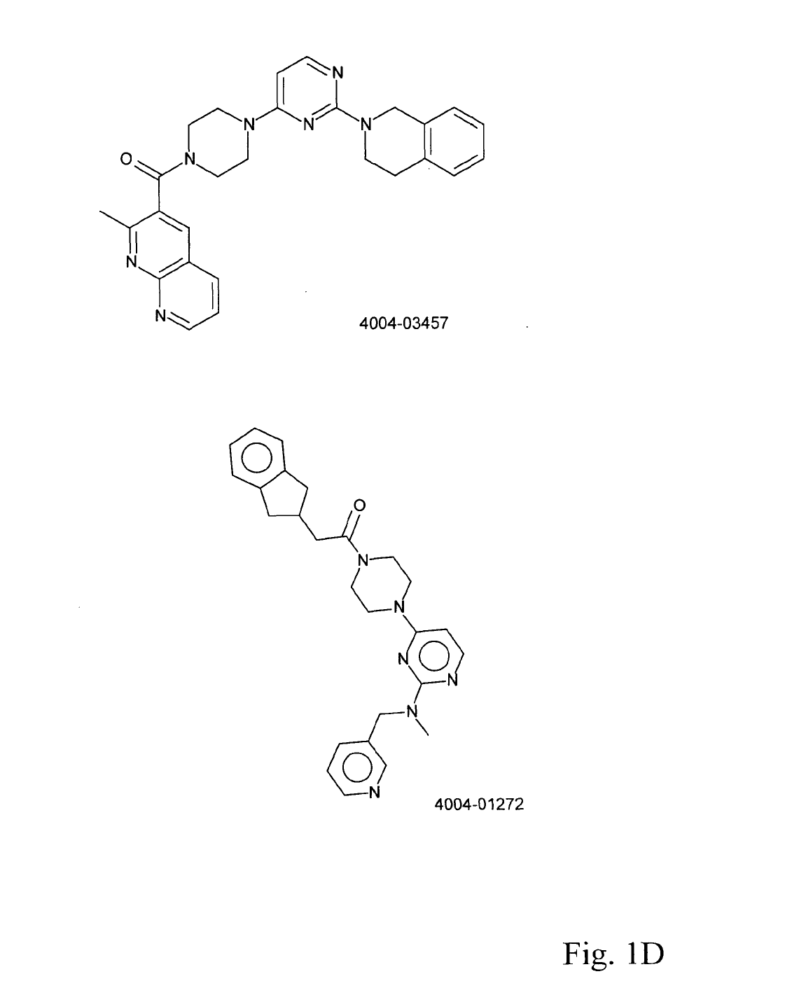

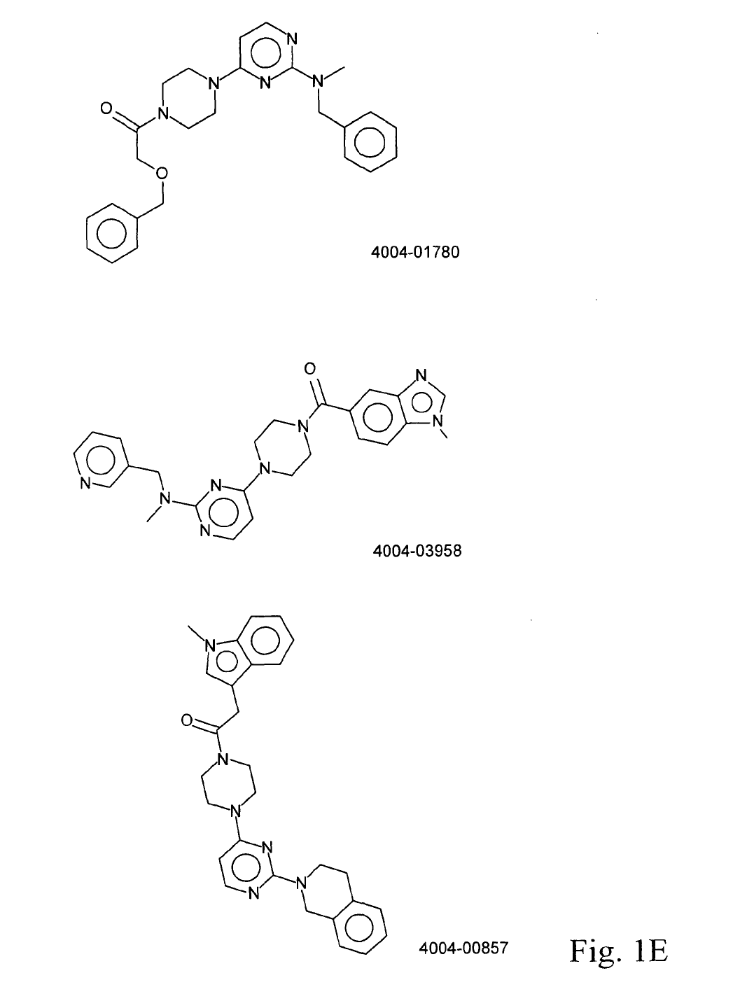

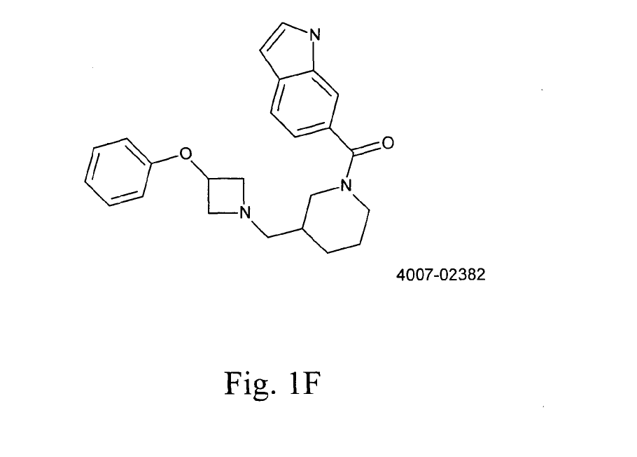

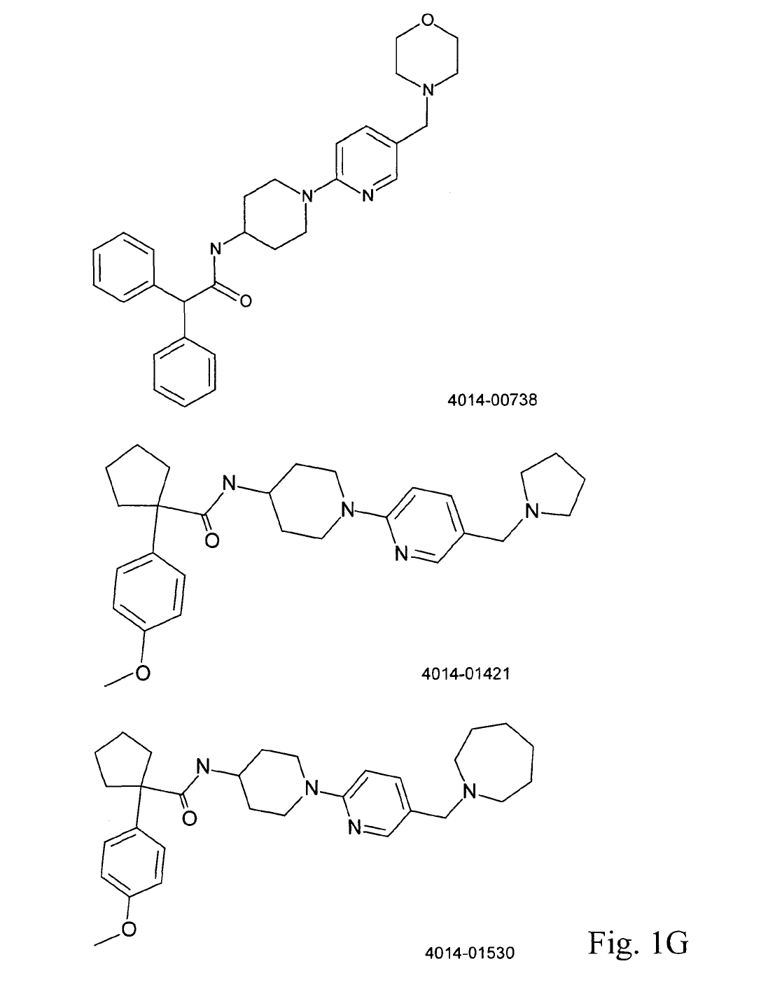

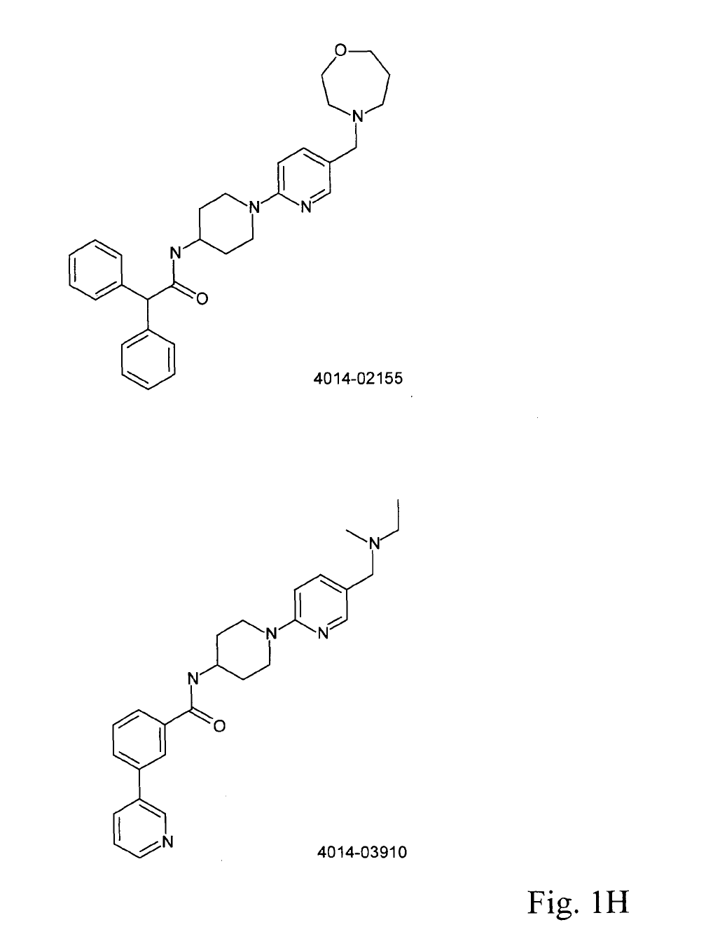

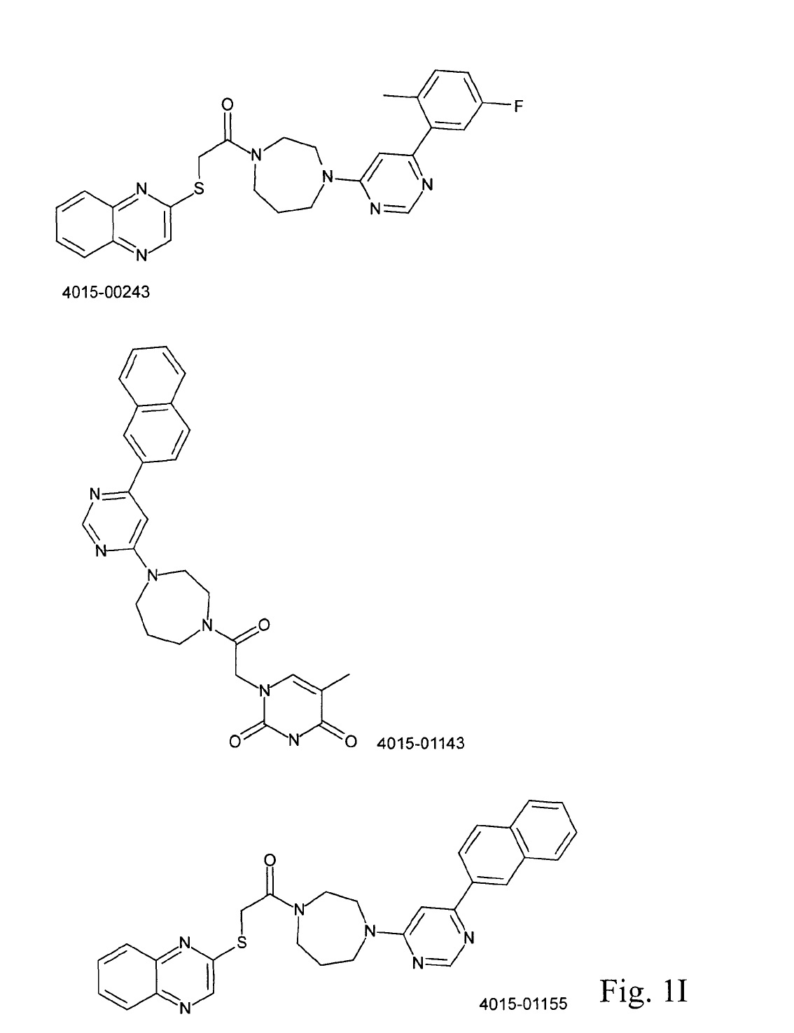

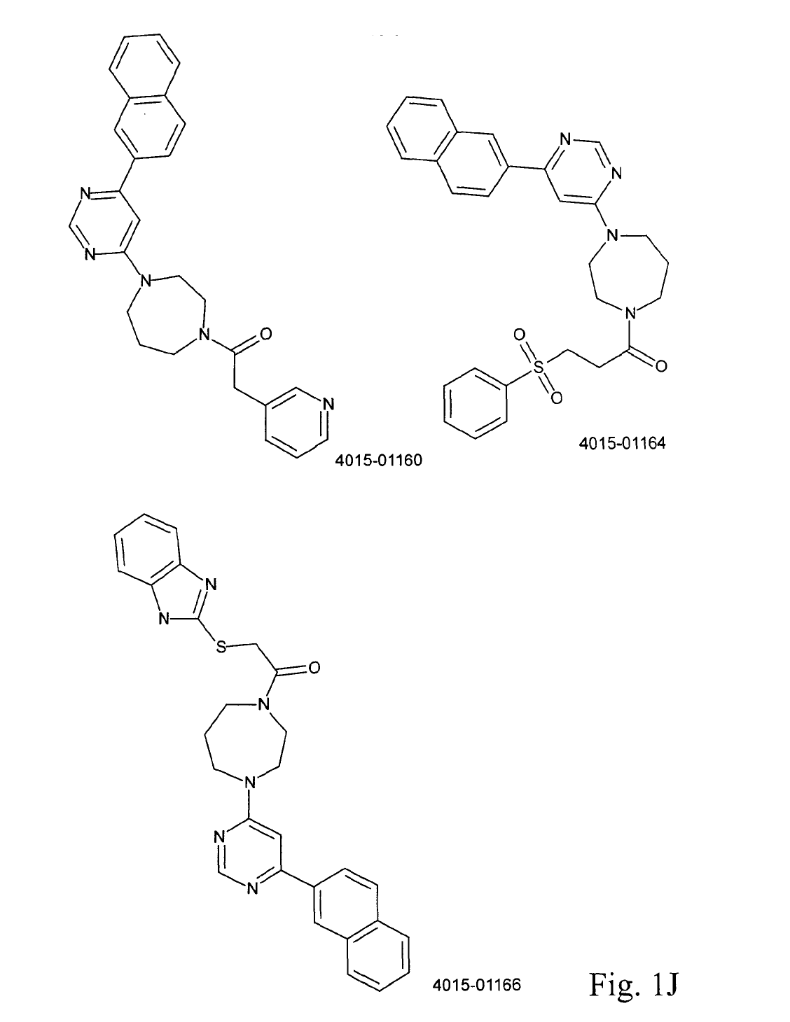

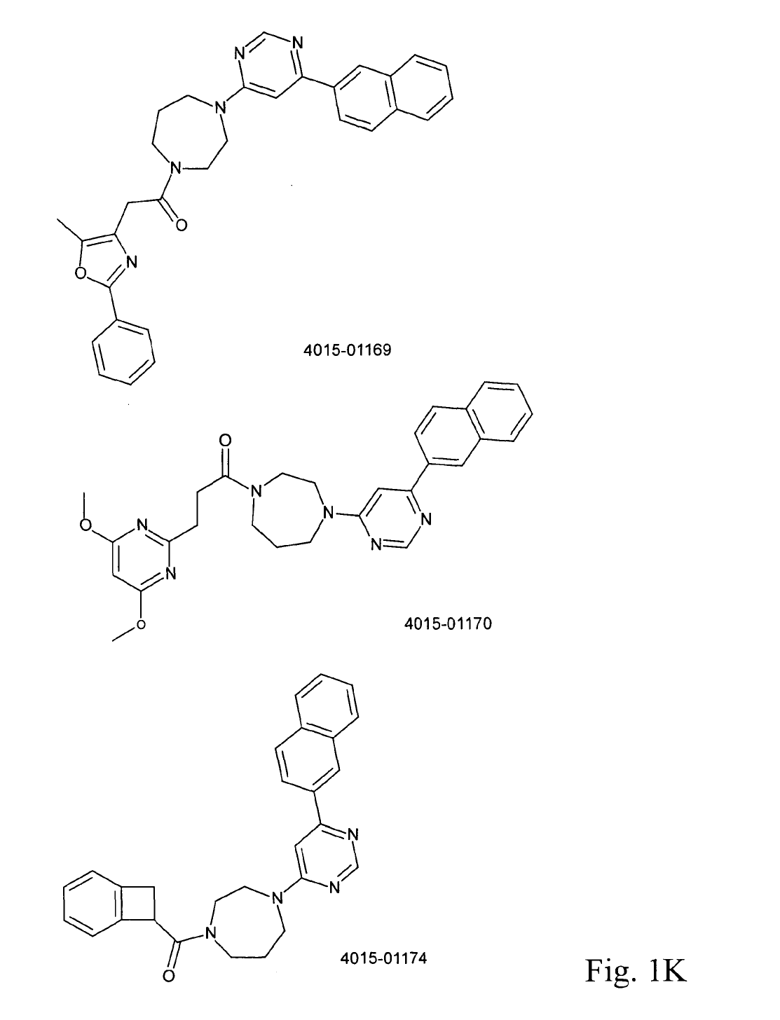

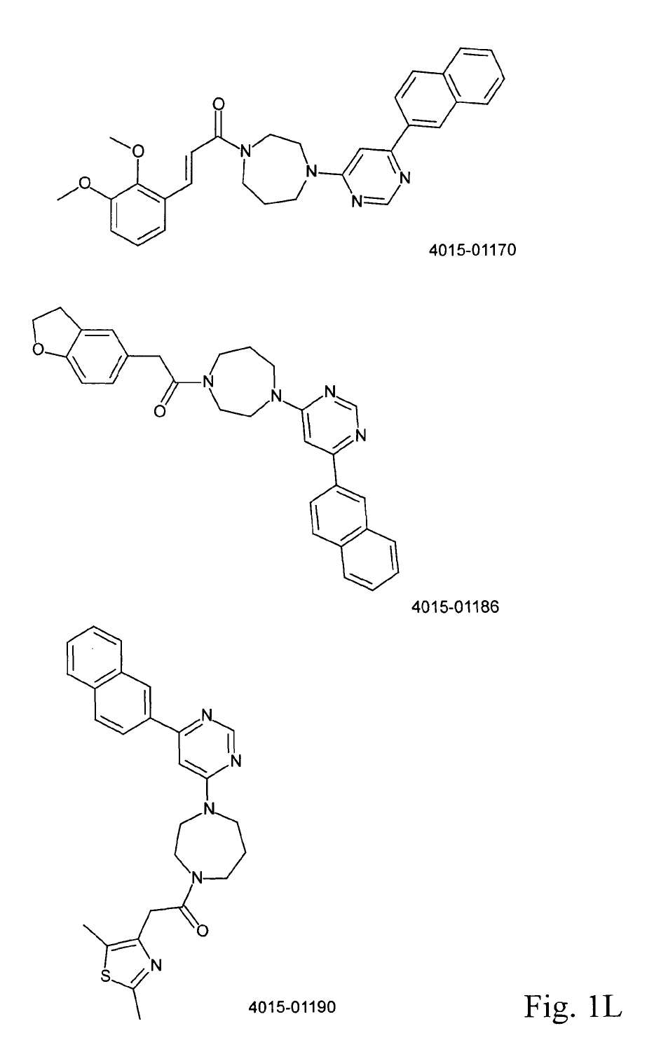

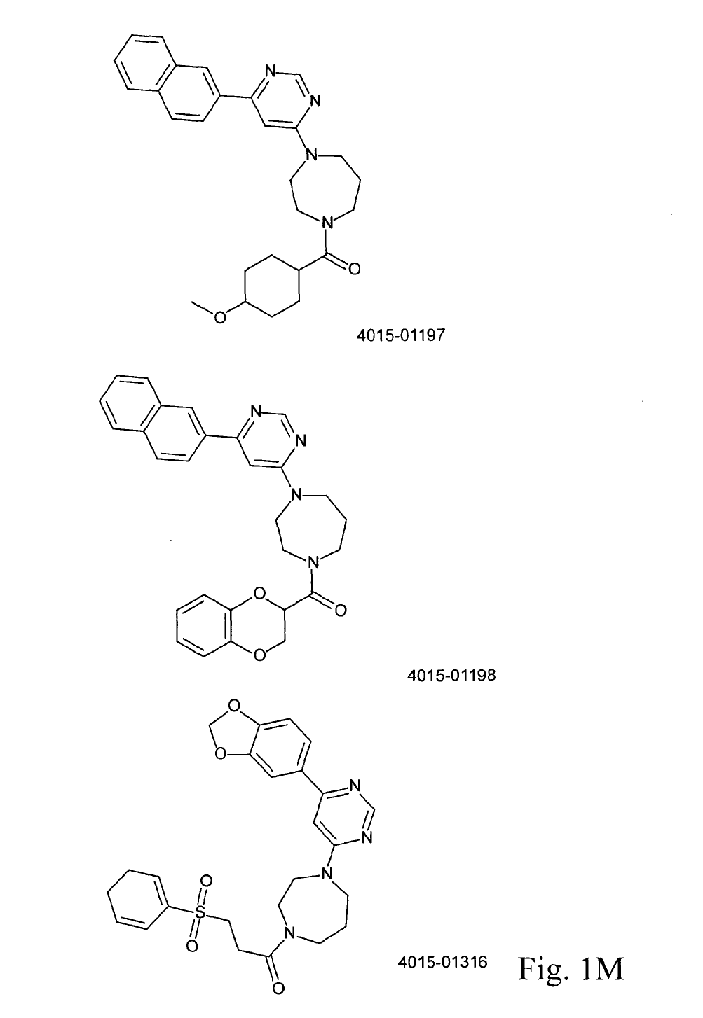

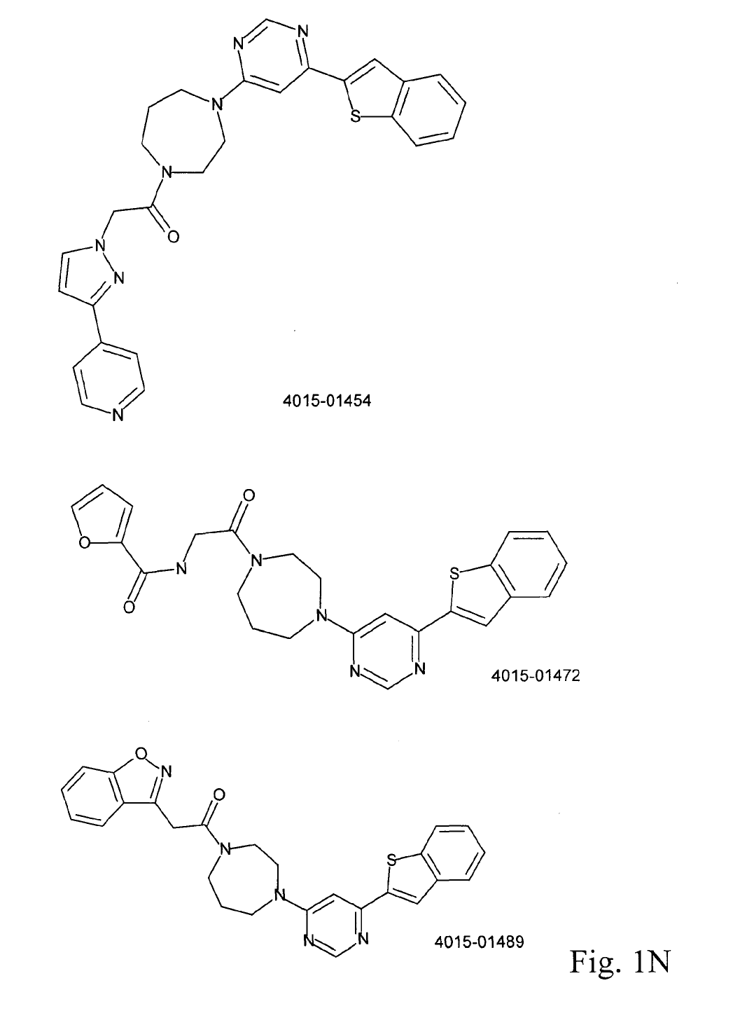

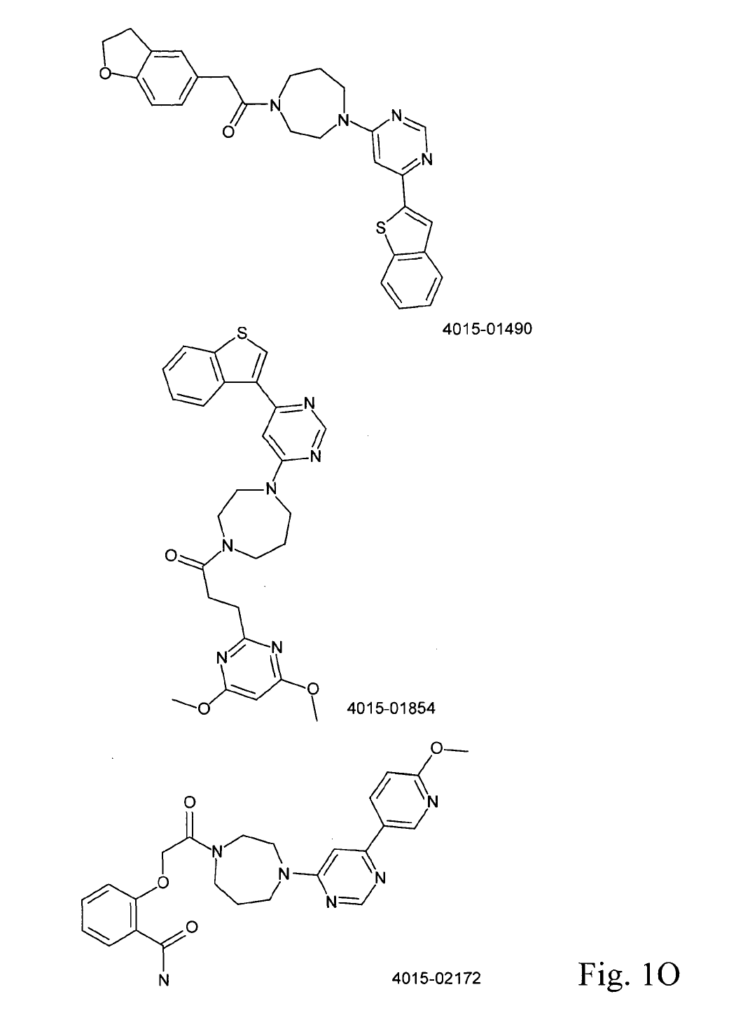

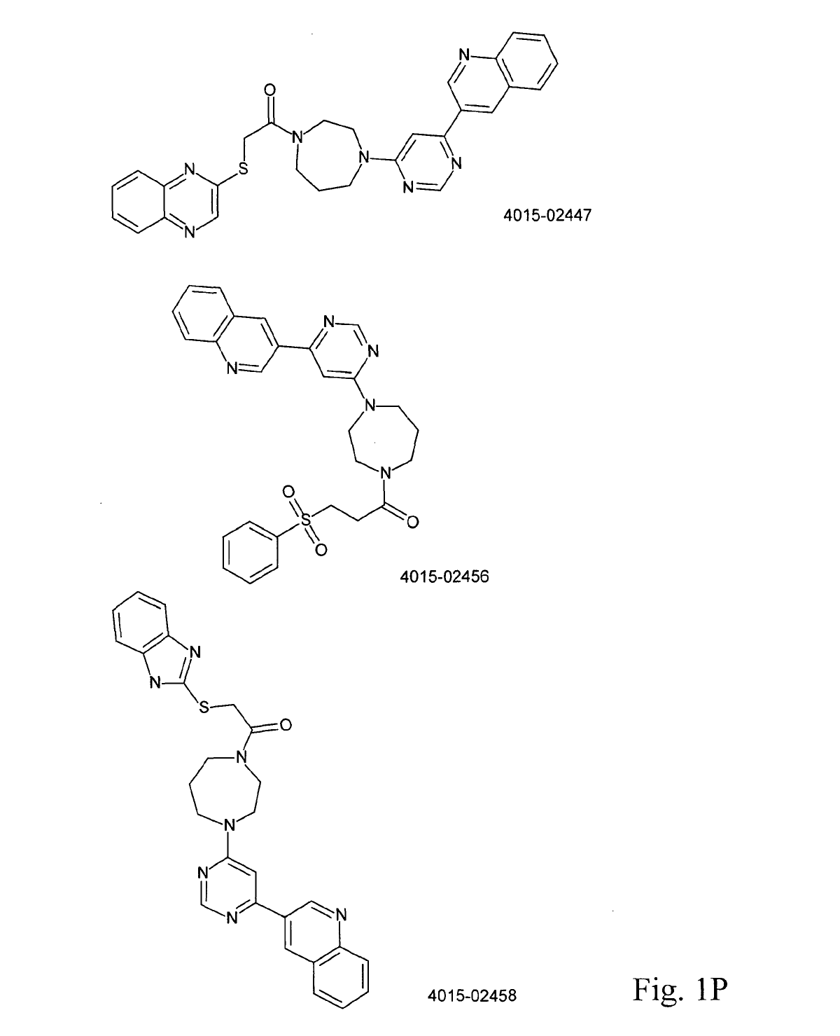

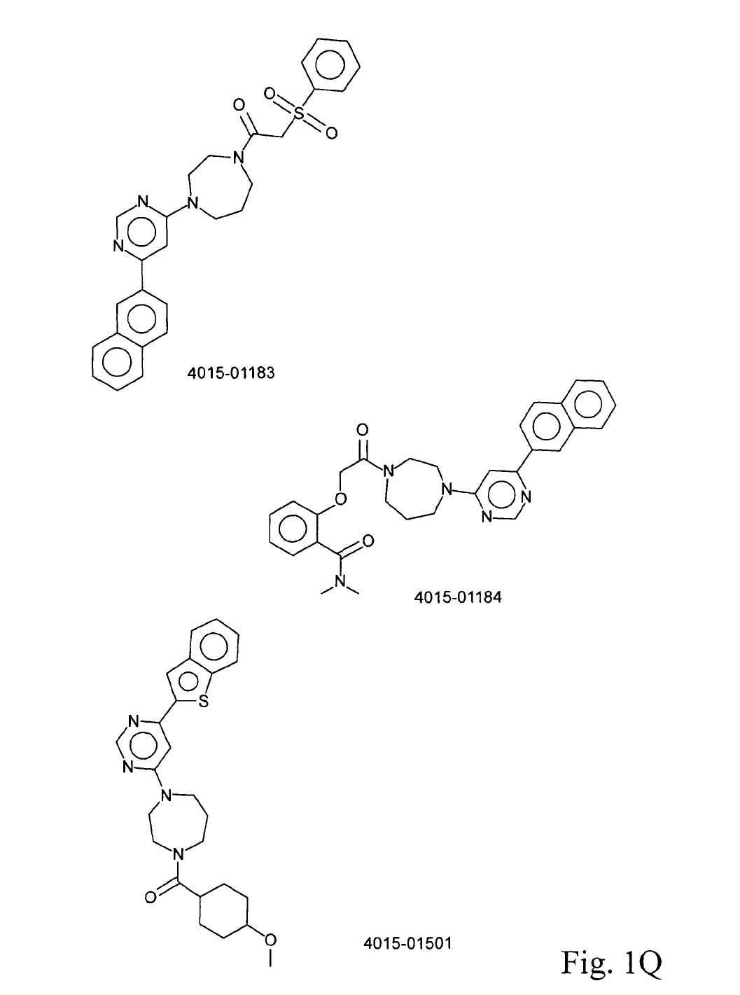

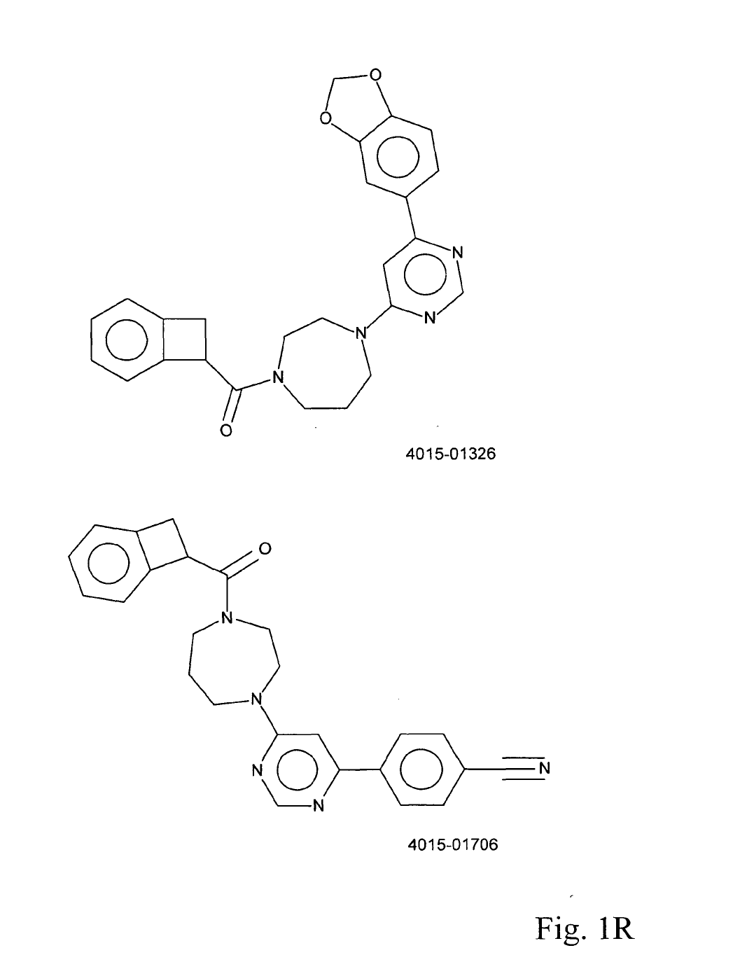

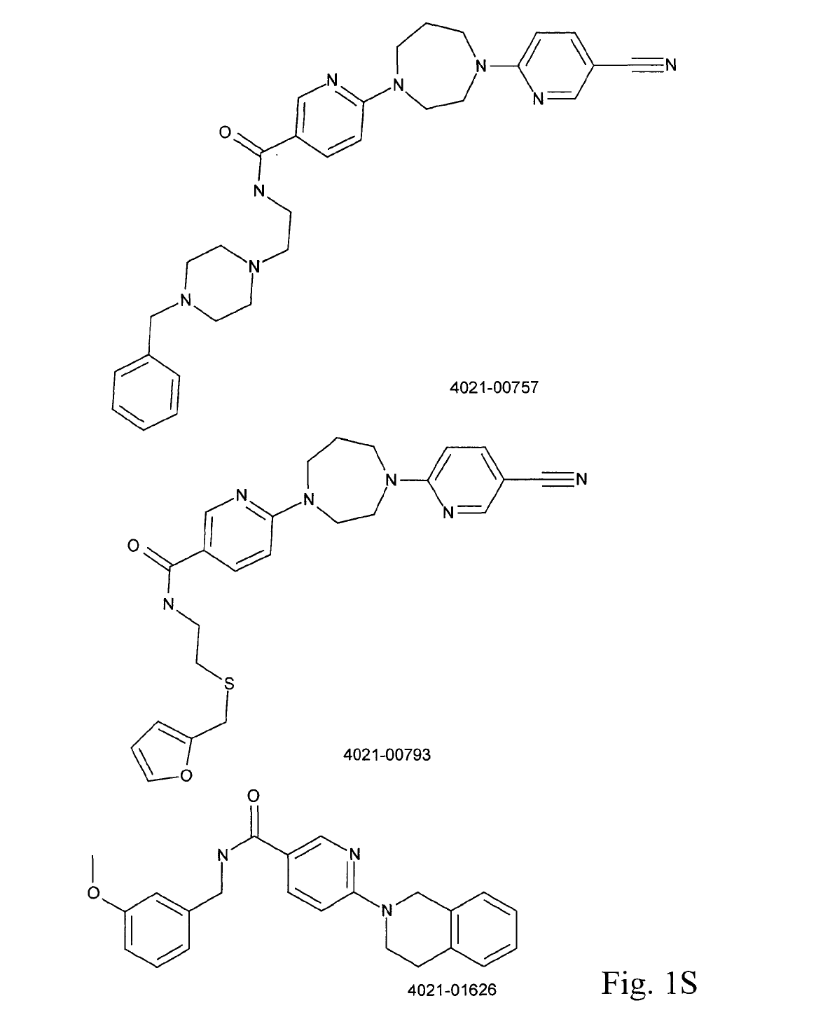

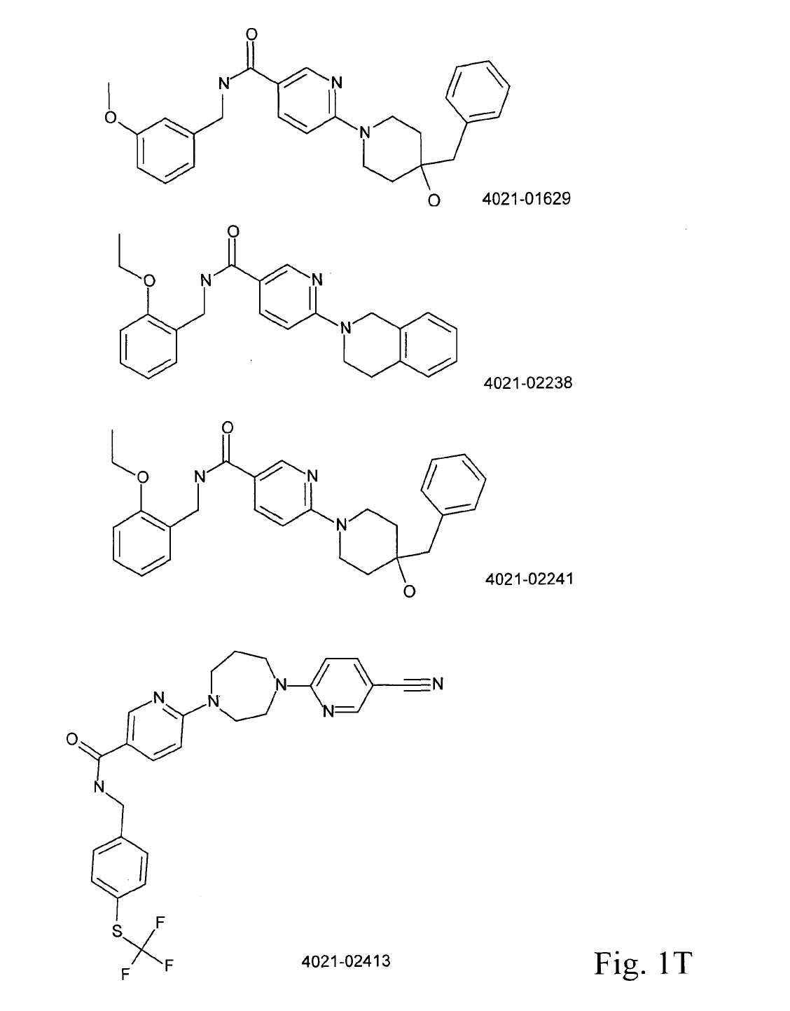

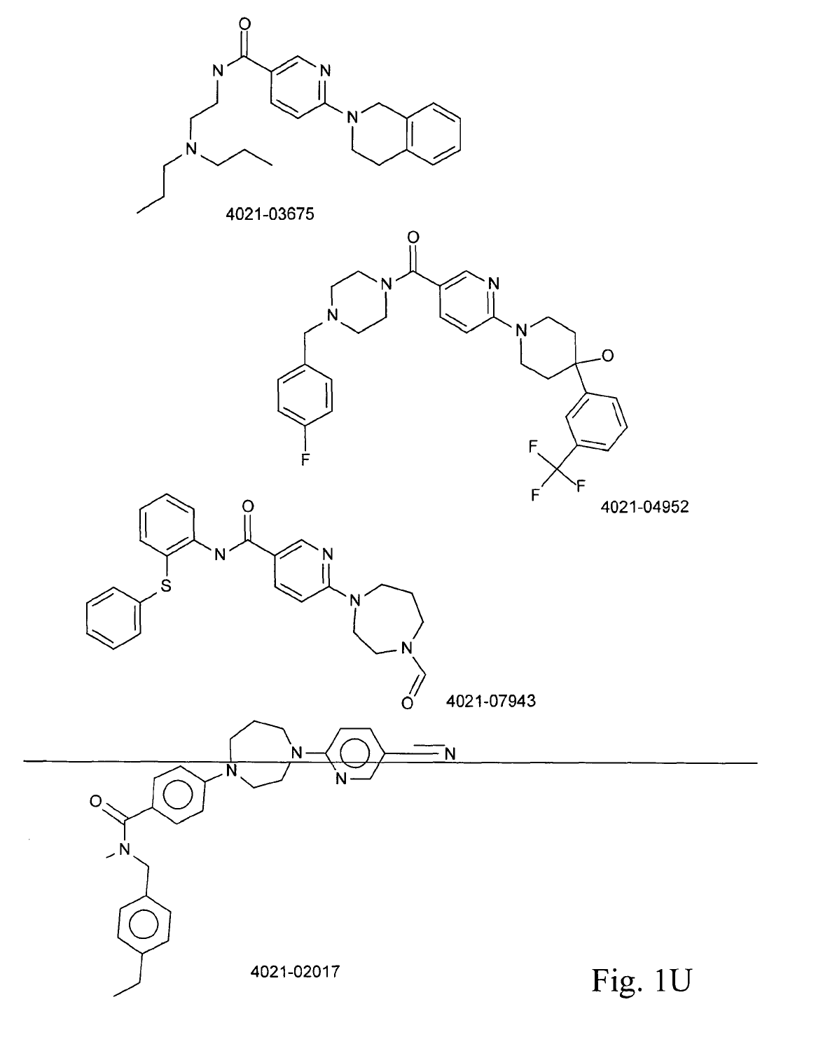

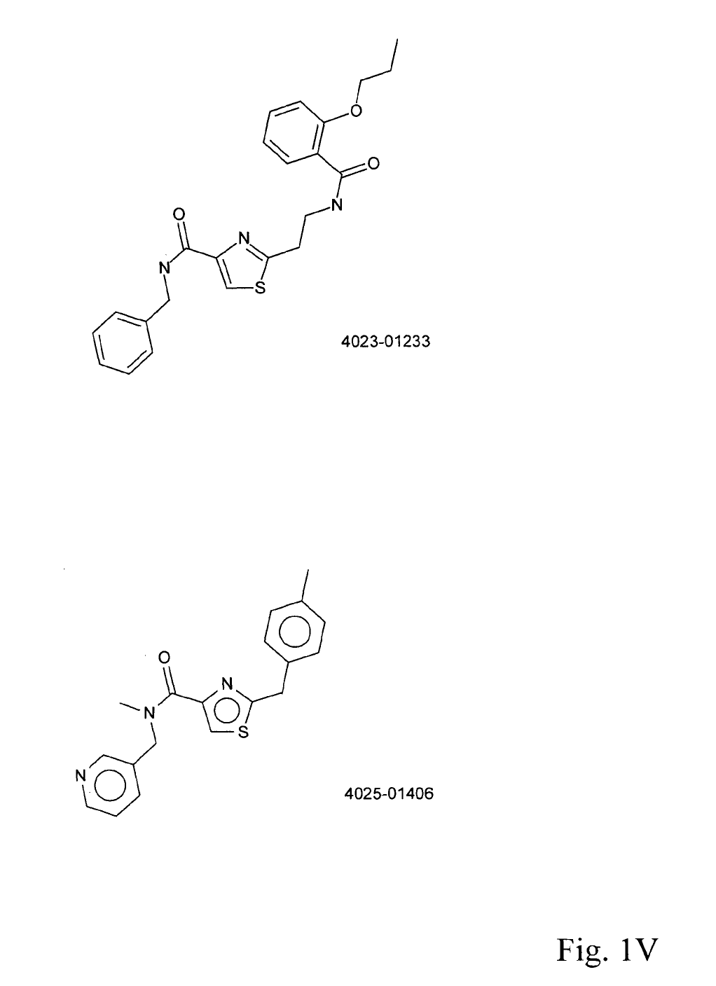

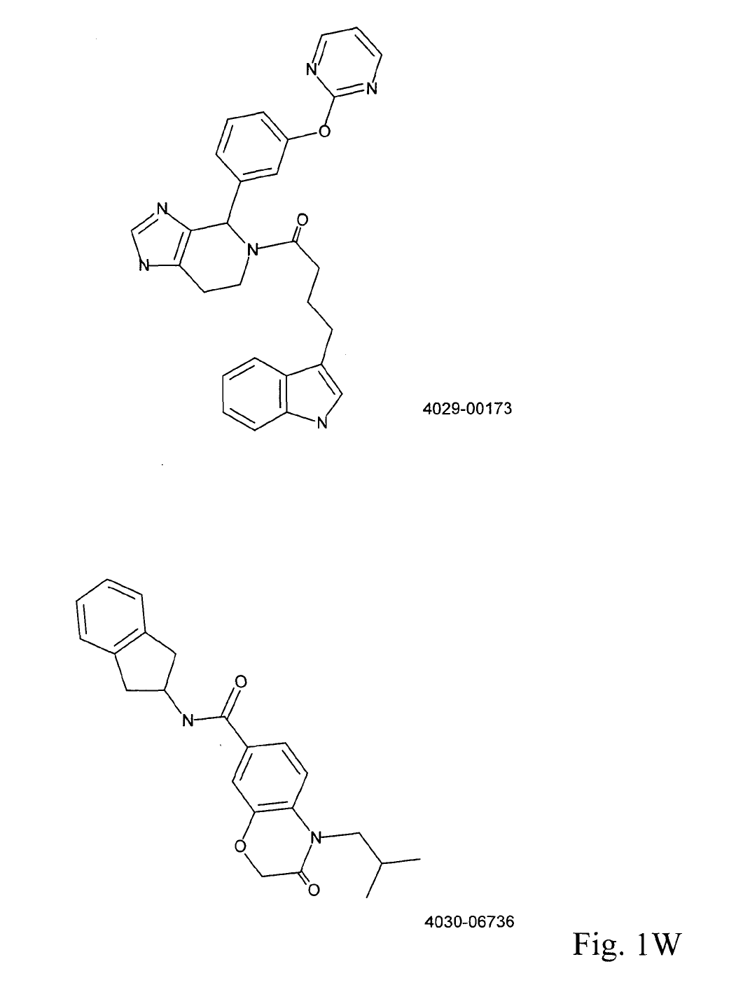

The competition assay described below was the assay used to test the compounds depicted in FIG. 1 and to generate the binding data given in FIG. 2.

The assay can be performed as follows: cells plated as described in Example 1 are exposed simultaneously to a fixed concentration of tritiated cyclopamine, e.g., 10 nM, and a concentration of test compound. Test compound is applied to different wells such that a wide range of concentrations is evaluated. Incubations are carried out over 60-90 minutes over ice or at room temperature. The cells are washed and extracted as above, and the amount of remaining tritiated cyclopamine determined as described in Example 1. By determining the amount of tritiated cyclopamine remaining specifically bound to the Smo receptor at the various test ligand concentrations, the affinity of the test ligands for the Smo receptor can be determined.

All documents and other information sources cited above are hereby incorporated in their entirety by reference.

1. A method of modulating Smoothened (Smo) receptor activity comprising administering to a patient in need thereof an amount of a compound of Formula I, or pharmaceutically acceptable salt thereof, 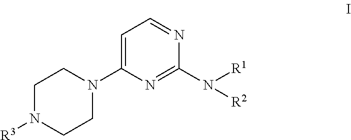
wherein R¹ is an linear or branched alkyl and R² is —(CH₂)ₙ— aryl and
n=1 to 4,
or R¹ and R² together with the nitrogen to which they are attached are 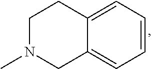
and
R³ is 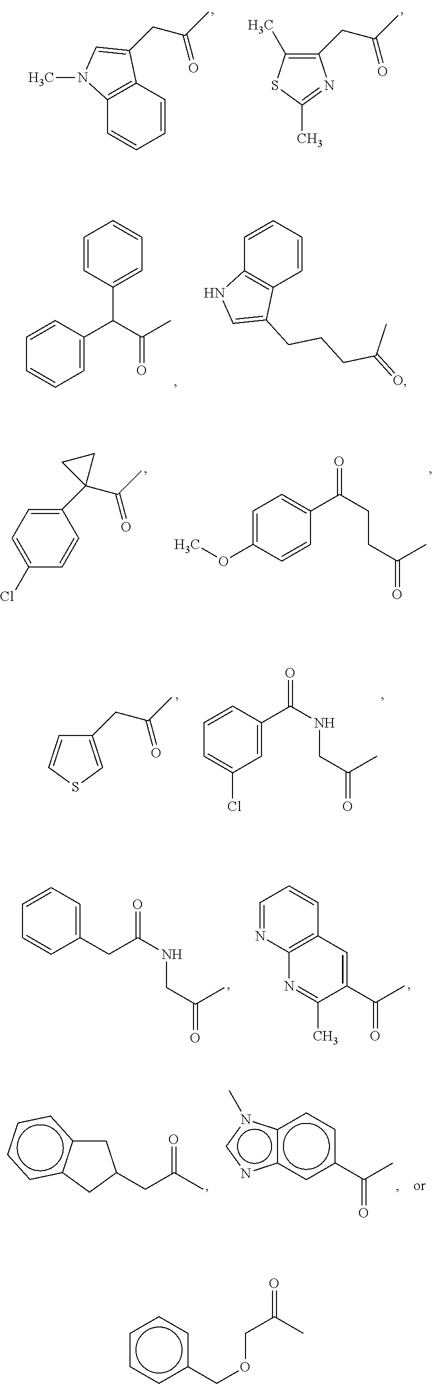
sufficient to effect said modulation.

2. A method of modulating Smo receptor activity comprising administering to a patient in need thereof an amount of a compound of Formula II, or pharmaceutically acceptable salt thereof, 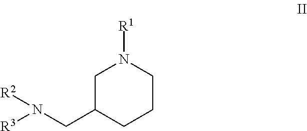
wherein R¹ is 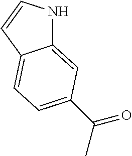
and R² and R³, together with the nitrogen to which they are attached are 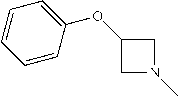
sufficient to effect said modulation.

3. A method of modulating Smo receptor activity comprising administering to a patient in need thereof an amount of a compound of Formula III, or pharmaceutically acceptable salt thereof, 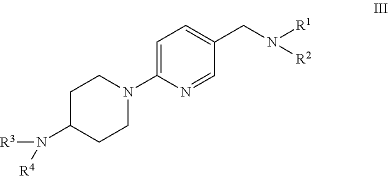
wherein R¹ and R², together with the nitrogen to which they are attached, are 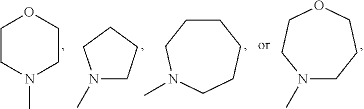
or
R¹ and R² are alkyl, and
R³ is H and R⁴ is 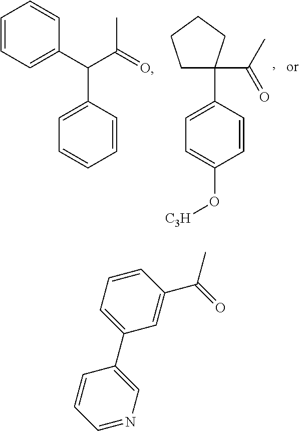
sufficient to effect said modulation.

4. A method of modulating Smo receptor activity comprising administering to a patient in need thereof an amount of a compound of Formula IV, or pharmaceutically acceptable salt thereof, 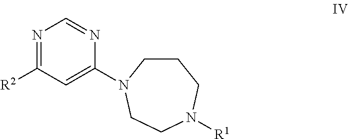
wherein R¹ is 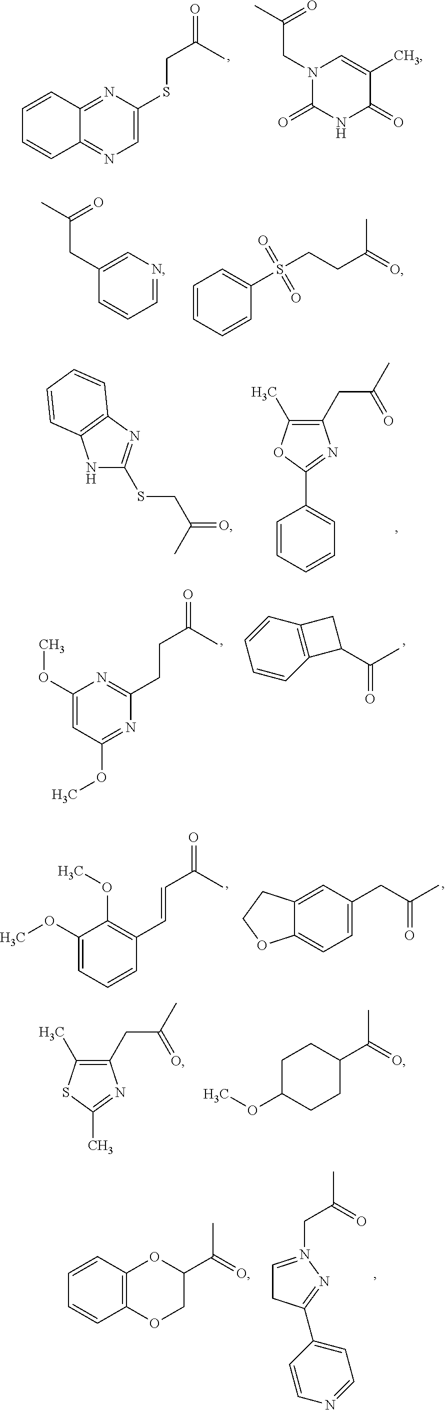 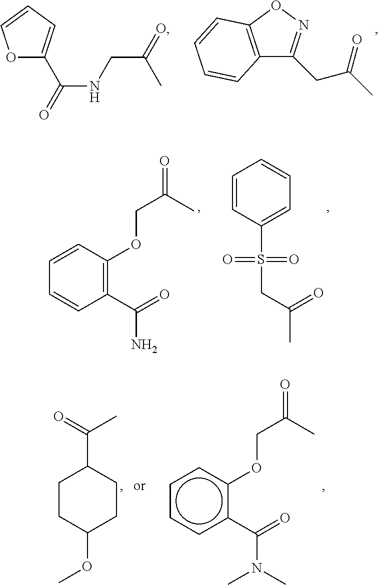
and
R² is 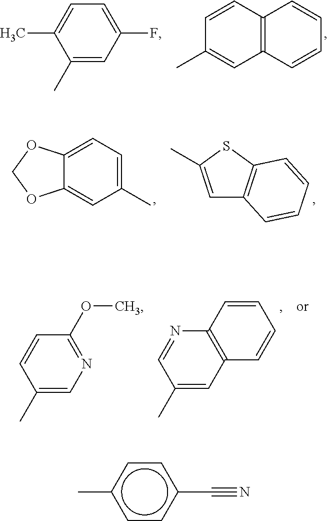
sufficient to effect said modulation.

5. A method of modulating Smo receptor activity comprising administering to a patient in need thereof an amount of a compound of Formula V, or pharmaceutically acceptable salt thereof, 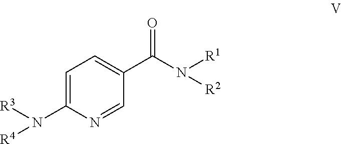
wherein R¹ is H and R² is 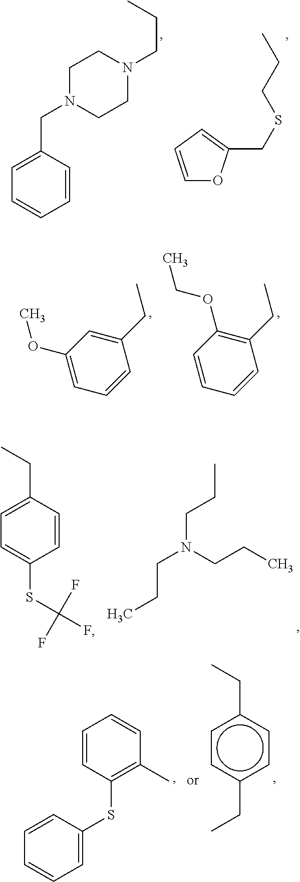
or R¹ and R² and the nitrogen to
which they are attached is 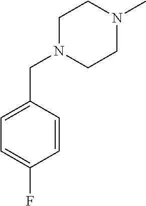
and
R³ and R⁴ and the nitrogen to which they are attached is 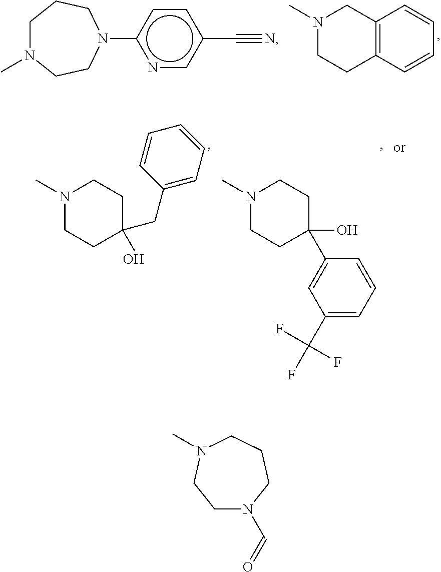
sufficient to effect said modulation.

6. A method of modulating Smo receptor activity comprising administering to a patient in need thereof an amount of a compound of Formula VI, or pharmaceutically acceptable salt thereof, 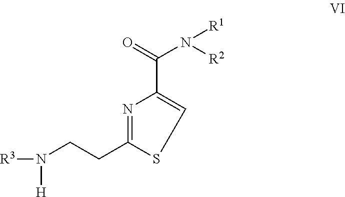
wherein R¹ is H and R² is 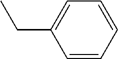
and 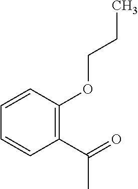
R³ is
sufficient to effect said modulation.

7. A method of modulating Smo receptor activity comprising administering to a patient in need thereof an amount of a compound of Formula VII, or pharmaceutically acceptable salt thereof, 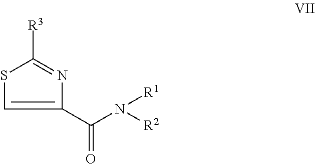
wherein R¹ is CH₃, and 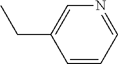
R² is
and R³ is 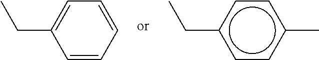
sufficient to effect said modulation.

8. A method of modulating Smo receptor activity comprising administering to a patient in need thereof an amount of a compound of Formula VIII, or pharmaceutically acceptable salt thereof, 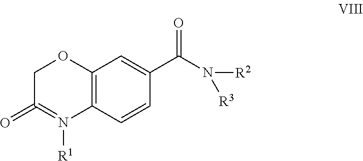
wherein R¹ is 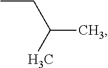
R² is H and R³ is 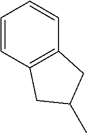
sufficient to effect said modulation.

9. The method according to claim 1 wherein said patient is a cancer patient and administration of said compound effects treatment of said cancer.

10. The method according to claim 9 wherein said cancer is, an adenocarcinoma of the pancreas, prostate, breast, stomach, esophagus or biliary tract; a medulloblastoma or glioma; a small-cell lung cancer; a basal cell carcinoma; a rhabdomyosarcoma; a urothelial carcinoma; a squamous cell carcinoma of the oral cavity; or a hepatocellular carcinoma.

11. The method according to claim 1 wherein said patient bears a wound and administration of said compound simulates healing of said wound.

12. A composition comprising a pharmaceutically acceptable carrier, excipient or diluent and a compound of Formula I, or pharmaceutically acceptable salt thereof, 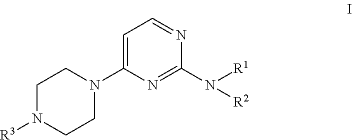
wherein R¹ is an linear or branched alkyl and R² is —(CH₂)ₙ— aryl and
n=1 to 4,
or R¹ and R² together with the nitrogen to which they are attached are 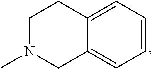
and
R³ is

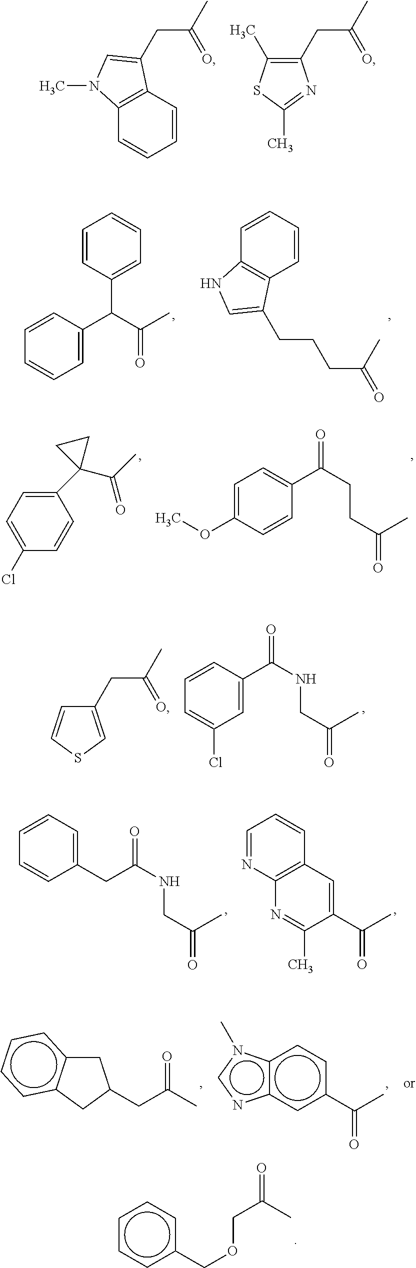

13. A composition comprising a pharmaceutically acceptable carrier, excipient or diluent and a compound of Formula II, or pharmaceutically acceptable salt thereof, 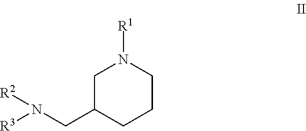
wherein R¹ is 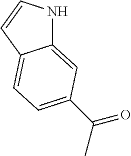
and R² and R³, together with the nitrogen
to which they are attached are

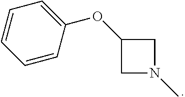

14. A composition comprising a pharmaceutically acceptable carrier, excipient or diluent and a compound of Formula III, or pharmaceutically acceptable salt thereof, 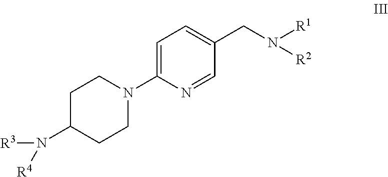
wherein R¹ and R², together with the nitrogen to which they are attached, are 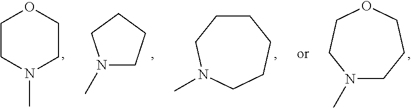
or
R¹ and R² are alkyl, and 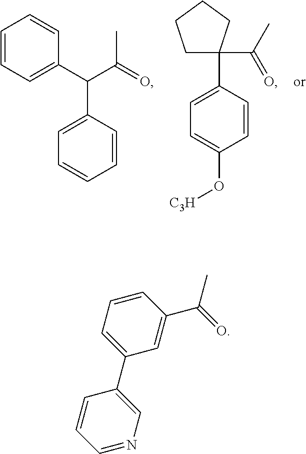
R³ is H and R⁴ is

15. A composition comprising a pharmaceutically acceptable carrier, excipient or diluent and a compound of Formula IV, or pharmaceutically acceptable salt thereof, 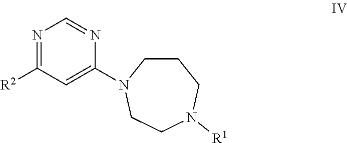
wherein R¹ is 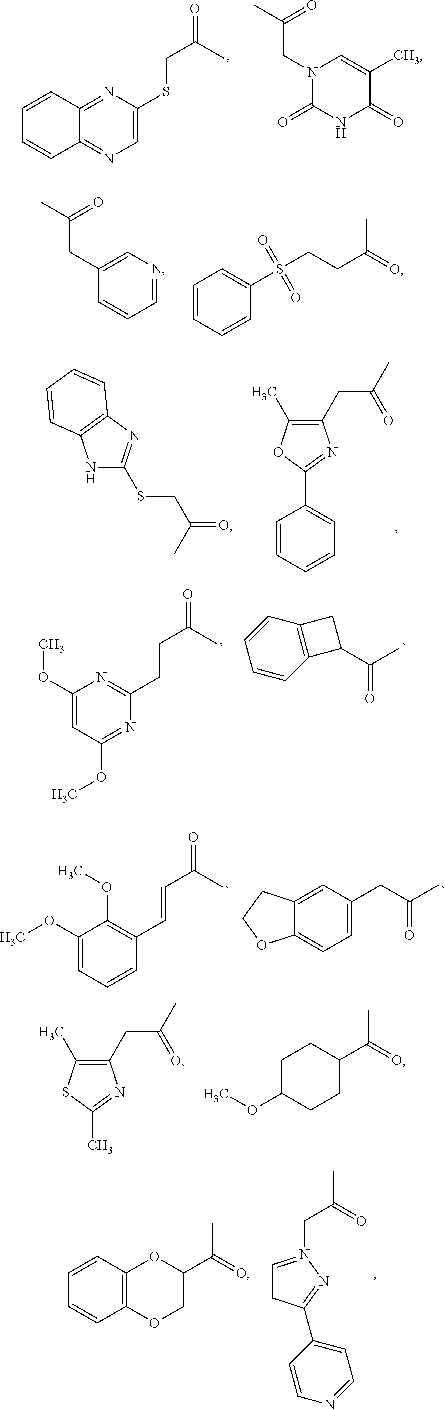 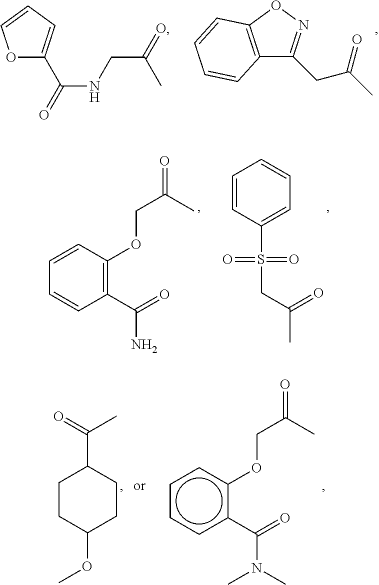
and
R² is

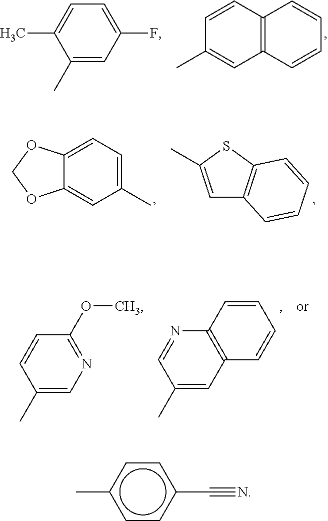

16. A composition comprising a pharmaceutically acceptable carrier, excipient or diluent and a compound of Formula V, or pharmaceutically acceptable salt thereof, 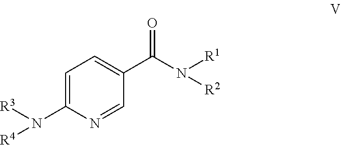
wherein R¹ is H and R² is 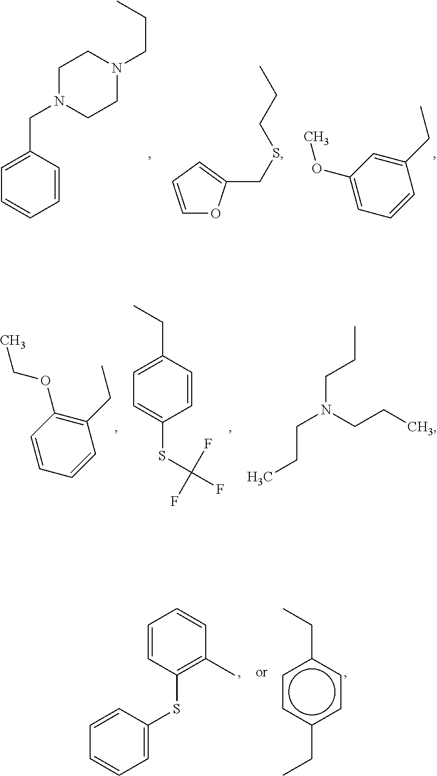
or R¹ and R² and the nitrogen to which they are attached is 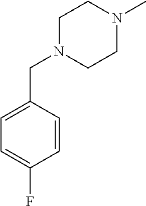
and
R³ and R⁴ and the nitrogen to which they are attached is

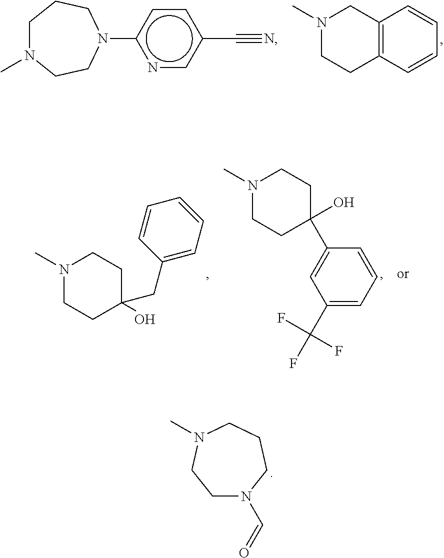

17. A composition comprising a pharmaceutically acceptable carrier, excipient or diluent and a compound of Formula VI, or pharmaceutically acceptable salt thereof, 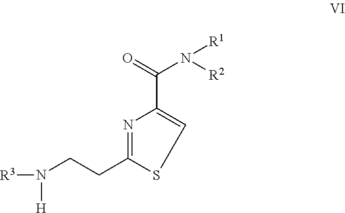
wherein R¹ is H and R² is 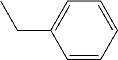
and
R³ is

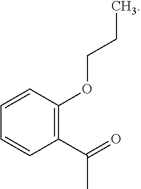

18. A composition comprising a pharmaceutically acceptable carrier, excipient or diluent and a compound of Formula VII, or pharmaceutically acceptable salt thereof, 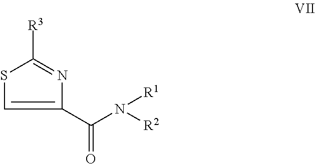
wherein R¹ is CH₃, and
R² is 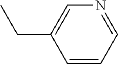
and R³ is

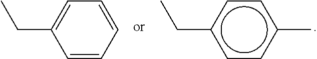

19. A composition comprising a pharmaceutically acceptable carrier, excipient or diluent and a compound of Formula VIII, or pharmaceutically acceptable salt thereof, 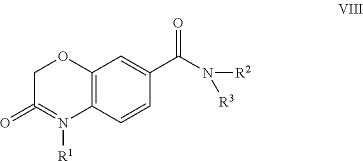
wherein R¹ is 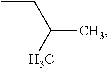
R² is H and R³ is

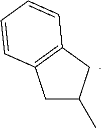

20. The composition according to claim 12 wherein said composition is in dosage unit form or is in the form of a sterile solution.
